(19) United States
(12) Patent Application Publication
(10) Pub. No.: US 2011/0151258 A1
(43) Pub. Date: Jun. 23, 2011

(54) PREPARATION OF RANOLAZINE

(75) Inventors: Raghupathi Reddy Anumula, Hyderabad (IN); Goverdhan Gilla, Hyderabad (IN); Sampath Aalla, Hyderabad (IN); Lokeswara Rao Madivada, Krishna (IN); Prabhaker Macherla, Hyderabad (IN); Srinivas Kurella, Nalgonda (IN); Kavitha Charagondla, Nalgonda (IN); Ramamurthy Kasula, Nellore (IN); Rajagopala Rao Mandadapu, Guntur (IN); Krishaniah Charagondla, Nalgonda (IN); Malati Vakamulla, Srikakulam (IN); Durga Prasad Janaki Bhavanipurapu, Krishna (IN)

(73) Assignees: DR. REDDY'S LABORATORIES LTD., Hyderabad (IN); DR. REDDY'S LABORATORIES, INC., Bridgewater, NJ (US)

(21) Appl. No.: 13/036,520

(22) Filed: Feb. 28, 2011

Related U.S. Application Data
(63) Continuation of application No. PCT/US2009/055367, filed on Aug. 28, 2009.
(60) Provisional application No. 61/122,467, filed on Dec. 15, 2008.
(60) Provisional application No. 61/148,225, filed on Jan. 29, 2009.

(30) Foreign Application Priority Data
Aug. 28, 2008 (IN) 2107/CHE/2008
Oct. 31, 2008 (IN) 2663/CHE/2008
Nov. 24, 2008 (IN) 2916/CHE/2008

(51) Int. Cl. B32B 5/16; C07D 295/15

(52) U.S. Cl. 428/402; 544/400

(57) ABSTRACT

Preparation of ranolazine and intermediates thereof, for use in pharmaceutical compositions comprising ranolazine.

### INTRODUCTION

Aspects of the present application relate to ranolazine, intermediates thereof, processes for the preparation of ranolazine and intermediates thereof, and pharmaceutical compositions comprising ranolazine.

Ranolazine is a racemic mixture having the chemical names: 1-piperazineacetamide, N-(2,6-dimethylphenyl)-4-[2-hydroxy-3-(2-methoxyphenoxy)propyl]-, (±)-; or (RS)—N-(2,6-dimethylphenyl)-2-[4-[2-hydroxy-3-(2-methoxyphenoxy)propyl]piperazin-1-yl]acetamide. It has the structure of Formula (I).

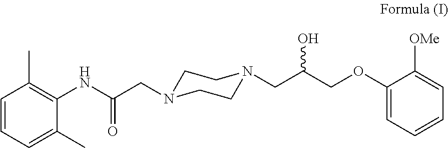

Ranolazine is prescribed for the treatment of chronic angina.

U.S. Pat. No. 4,567,264 (“the '264 patent”) discloses ranolazine and pharmaceutically acceptable esters and acid addition salts thereof. The '264 patent also discloses two processes for the synthesis of ranolazine.

The first process disclosed in the '264 patent involves reacting 2-methoxy phenol with an excess of epichlorohydrin, in the presence of sodium hydroxide, and in a mixture of water and dioxane, to provide 1-(2-methoxyphenoxy)-2,3-epoxypropane. This compound is reacted with piperazine in ethanol, at ambient temperature, for two days to provide 1-[3-(2-methoxyphenoxy)-2-hydroxypropyl]-piperazine. The 1-[3-(2-methoxyphenoxy)-2-hydroxypropyl]-piperazine is reacted with [(2,6-dimethylphenyl)aminocarbonylmethyl]-chloride in dimethylformamide, to provide ranolazine as an oil, which is further purified by chromatography using 5% methanol in methylene chloride to provide ranolazine as a yellow oily compound. The compound is crystallized using hydrochloric acid in methanol.

The second process disclosed in the '264 patent involves reacting [(2,6-dimethylphenyl)aminocarbonylmethyl]-chloride with piperazine in ethanol, to provide 1-[(2,6-dimethylphenyl)aminocarbonylmethyl] piperazine. This compound is reacted with 1-(2-methoxyphenoxy)-2,3-epoxypropane in a mixture of methanol and toluene. After completion of the reaction, the reaction solution is evaporated, chromatographed, and the product converted into its dihydrochloride salt using excess hydrochloric acid in methanol.

The '264 patent also discloses a process for providing ranolazine as a free base, by treating ranolazine dihydrochloride salt with ammonium hydroxide in water.

The process for the preparation of 1-(2-methoxyphenoxy)-2,3-epoxypropane disclosed in the '264 patent involves the use of dioxane as a solvent medium, which is not preferable for large scale production.

Moreover, the present inventors have discovered that the processes of the '264 patent may result in the formation of up to 43% of the dimer impurity of Formula (IIa).

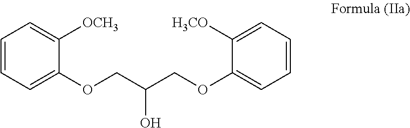

International Application Publication No. WO 2006/008753 A1 discloses a process for the preparation of ranolazine by reacting 1-(2-methoxyphenoxy)-2,3-epoxpropane with anhydrous piperazine in methanol, to get 1-[3-(2-methoxyphenoxy)-2-hydroxypropyl]-piperazine. This compound is reacted with [(2,6-dimethylphenyl)aminocarbonylmethyl] chloride in anhydrous potassium carbonate and sodium iodide, in dimethylformamide, to get ranolazine dihydrochloride. The publication also discloses preparation of ranolazine free base by treating its dihydrochloride salt with liquor ammonia, in a mixture of water and acetone.

International Application Publication No. WO 2008/047388 A2 discloses a process for the preparation of ranolazine, by reacting 2-methoxyphenol with epichlorohydrin in the presence of a solution of sodium hydroxide in water and tetrabutyl ammonium bromide in toluene, to get 1-methoxy-2-(oxiranylmethoxy) benzene having a purity of 84.21% and a dimer impurity of 7.59%. The compound is subjected to high vacuum distillation at 130-150° C., to get 1-methoxy-2-(oxiranylmethoxy)benzene having a purity of 96.10%. This compound is reacted with N-(2,6-dimethylphenyl)-1-piperazine acetamide in toluene and, after maintaining the reaction mixture for 5 hours at 120° C., the reaction mixture is acidified with dilute hydrochloric acid. The aqueous layer pH is adjusted to 7-8 with sodium bicarbonate and the compound is extracted from the aqueous layer with methylene chloride. The solvent from the organic layer is evaporated to get crude ranolazine, and the crude compound is crystallized from ethanol to get ranolazine having a purity of 99.58%.

Roles of particle sizes in formulation processing, such as mixing and de-mixing, content uniformity, compressibility, dissolution, and bioavailability are well known. Particle sizes play a critical role for active pharmaceutical ingredients that are BCS Class II or Class IV. For low dose drugs, particle sizes become particularly critical for attaining proper content uniformity and release profiles. For high dose drugs, particle sizes are important for compressibility, compactness of the final dosage form, and release profiles. Further complexity arises if the final dosage form desired is a modified release dosage form. For example, since ranolazine has low solubility, but is offered in a high dose from a controlled release formulation, defining suitable particle sizes that deliver reproducible formulations and low variations in release rate from individual dosage units is very difficult. Desirable formulation characteristics can be achieved by defining the right particle sizes of the ranolazine, apart from other aspects of the formulation. However, there is no predictive tool that help identify the desirable particle sizes of active pharmaceuticals. Thus, a great challenge lies in front of a formulator designing a dosage form.

There remains a need to provide processes for the preparation of ranolazine that are cost-effective and environmentally friendly, and that avoid the use of high vacuum distillation and column chromatography. Further, the present inventors have discovered that a solid form of ranolazine may be directly isolated by the process of the present application without conversion into an acid addition salt and, in turn, the conversion of the acid addition salt back into ranolazine.

### SUMMARY

In aspects, the present application provides processes for the preparation of ranolazine of Formula (I) or a pharmaceutically acceptable salt thereof, embodiments including one or more of the following steps, individually or in the sequence recited:

(a) reacting 2-methoxy phenol with epichlorohydrin in the presence of a base to provide 1-(2-methoxyphenoxy)-2,3-epoxypropane of Formula (II),

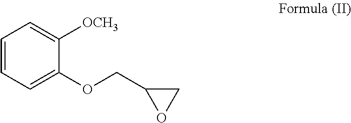

wherein the base is added to the reaction mixture in more than one portion;

(b) reacting 2,6-dimethylaniline with chloroacetylchloride in the presence of a base to provide [(2,6-dimethylphenyl)aminocarbonylmethyl]-chloride of Formula (III);

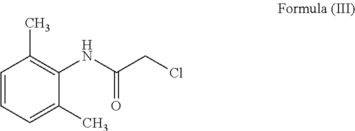

(c) reacting [(2,6-dimethylphenyl)aminocarbonylmethyl]-chloride of Formula (III) with piperazine in a solvent, to provide N-(2,6-dimethylphenyl)-1-piperazine acetamide of Formula (IV);

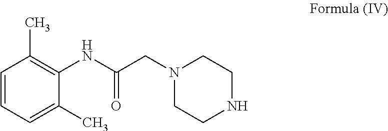

(d) reacting 1-(2-methoxyphenoxy)-2,3-epoxypropane of Formula (II) with N-(2,6-dimethylphenyl)-1-piperazine acetamide of Formula (IV), to provide ranolazine of Formula (I); and

(e) isolating ranolazine of Formula (I) in solid form from the reaction mixture that is obtained in (d).

In embodiments, the present application provides processes for the preparation of ranolazine of Formula (I) or a pharmaceutically acceptable salt thereof, which include one or more of the following steps, individually or in the sequence recited:

(a) reacting 2-methoxyphenol with epichlorohydrin, in the presence of a base, to provide 1-(2-methoxyphenoxy)-2,3-epoxypropane of Formula (II),

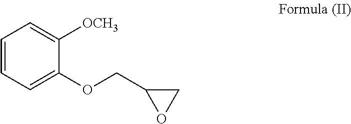

wherein the base is added to the reaction mixture in small portions;

(b) reacting 1-(2-methoxyphenoxy)-2,3-epoxypropane of Formula (II) with piperazine in a solvent to provide 1-[3-(2-methoxyphenoxy)-2-hydroxypropyl]-piperazine of Formula (V);

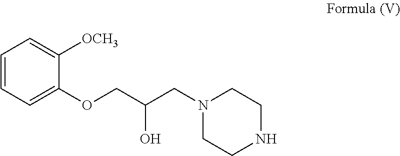

(c) reacting 1-[3-(2-methoxyphenoxy)-2-hydroxypropyl]-piperazine of Formula (V) with [(2,6-dimethylphenyl)aminocarbonylmethyl]-chloride of Formula (III), in a solvent, to provide ranolazine of Formula (I); and

(d) isolating ranolazine of Formula (I) in solid form from the reaction mixture that is obtained in (c).

In aspects, the present application provides processes for making ranolazine of Formula (I) in a solid form, an embodiment of which includes:

(a) reacting the compound of Formula (II) with the compound of Formula (IV); or

(b) reacting the compound of Formula (III) with the compound for Formula (V);

and isolating ranolazine of Formula (I) in solid form.

In embodiments, the present application provides N-(2,6-dimethylphenyl)-1-piperazine acetamide of Formula (IV), substantially free of piperazine.

In embodiments, the present application provides processes for the preparation of N-(2,6-dimethylphenyl)-1-piperazine acetamide of Formula (IV), substantially free of piperazine, including:

(i) providing a mixture containing N-(2,6-dimethylphenyl)-1-piperazine acetamide of Formula (IV) in a solvent;

(ii) adjusting pH to less than about 7 with an acid; and

(iii) adjusting pH of the reaction mass to greater than about 8 with a base and isolating N-(2,6-dimethylphenyl)-1-piperazine acetamide of Formula (IV), substantially free of piperazine.

In embodiments, the present application provides ranolazine having maximum particle sizes less than about 150 μm, or less than about 100 μm, or less than about 50 μm, or less than about 20 μm, or less than about 10 μm.

In embodiments, the present application provides ranolazine having bulk densities less than about 0.8 g/mL, less than about 0.5 g/mL, or less than about 0.3 g/mL.

In embodiments, the present application provides ranolazine having specific surface areas greater than about 0.1 m²/g, greater than about 0.5 m²/g, greater than about 1 m²/g, greater than about 2 m²/g, greater than about 3 m²/g, or greater than about 5 m²/g.

In embodiments, the present application provides pharmaceutical compositions prepared using ranolazine having maximum particle sizes less than about 150 μm, or less than about 100 μm, or less than about 50 μm, or less than about 20 μm, or less than about 10 μm, together with one or more pharmaceutically acceptable excipients.

In embodiments, the present application provides pharmaceutical compositions prepared using ranolazine having bulk densities less than about 0.8 g/mL, less than about 0.5 g/mL, or less than about 0.3 g/mL, together with one or more pharmaceutically acceptable excipients.

In embodiments, the present application provides pharmaceutical compositions prepared using ranolazine having specific surface areas greater than about 0.1 m²/g, greater than about 0.5 m²/g, greater than about 1 m²/g, greater than about 2 m²/g, greater than about 3 m²/g, or greater than about 5 m²/g, together with one or more pharmaceutically acceptable excipients.

In embodiments, the present application provides processes for the preparation of ranolazine, wherein the ranolazine is isolated without first forming a salt.

In embodiments, the present application provides processes for the preparation of ranolazine, wherein the processes do not include use of high vacuum distillation or column chromatography.

### BRIEF DESCRIPTION OF THE DRAWINGS

FIG. 1 is an illustration of a powder X-ray diffraction (PXRD) pattern of ranolazine prepared according to Example 6(A).

FIG. 2 is an illustration of a differential scanning calorimetry (DSC) thermogram of ranolazine prepared according to Example 6(A).

FIG. 3 is an illustration of a thermogravimetric analysis (TGA) curve of ranolazine prepared according to Example 6(A).

FIG. 4 is an illustration of a PXRD pattern of N-(2,6-dimethylphenyl)-1-piperazine acetamide having polymorphic crystalline Form A, prepared according to Example 21(A).

FIG. 5 is an illustration of an infrared (IR) absorption spectrum of N-(2,6-dimethylphenyl)-1-piperazine acetamide having polymorphic crystalline Form A, prepared according to Example 21(A).

FIG. 6 is an illustration of a DSC thermogram of N-(2,6-dimethylphenyl)-1-piperazine acetamide having polymorphic crystalline Form A, prepared according to Example 21(A).

FIG. 7 is an illustration of a TGA curve of N-(2,6-dimethylphenyl)-1-piperazine acetamide having polymorphic crystalline Form A, prepared according to Example 21(A).

FIG. 8 is an illustration of a PXRD pattern of N-(2,6-dimethylphenyl)-1-piperazine acetamide having polymorphic crystalline Form B, prepared according to Example 21(B).

FIG. 9 is an illustration of an IR absorption spectrum of N-(2,6-dimethylphenyl)-1-piperazine acetamide having polymorphic crystalline Form B, prepared according to Example 21(B).

FIG. 10 is an illustration of a DSC thermogram of N-(2,6-dimethylphenyl)-1-piperazine acetamide having polymorphic crystalline Form B, prepared according to Example 21(B).

FIG. 11 is an illustration of a TGA curve of N-(2,6-dimethylphenyl)-1-piperazine acetamide having polymorphic crystalline Form B, prepared according to Example 21(B).

FIG. 12 is a photomicrograph showing the particle shape of ranolazine of Formula (I) prepared according to Example 16.

FIG. 13 is a photomicrograph showing the particle shape of ranolazine of Formula (I) prepared according to Example 17.

FIG. 14 is a photomicrograph showing the particle shape of ranolazine of Formula (I) prepared according to Example 19.

### DETAILED DESCRIPTION

All percentages and ratios used herein are by weight of the total composition and all measurements made are at 25° C. and atmospheric pressure unless the context indicates otherwise. All temperatures are in degrees Celsius unless specified otherwise. As used herein, “comprising” means the elements recited, or their equivalent in structure or function, plus any other element or elements that are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. All ranges recited herein include the endpoints, including those that recite a range “between” two values. The terms “about,” “generally,” “substantially,”, and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

Where this document refers to a material, such as, for example, ranolazine, and the unique solid and/or crystalline forms, salts, solvates, and/or optical isomers thereof by reference to patterns, spectra, or other graphical data, it may do so by qualifying that they are “substantially” shown or depicted in a figure, or by one or more data points. It will be appreciated that patterns, spectra, and other graphical data may have features shifted in their positions, relative intensities, or other values due to a number of factors known to those of skill in the art. For example, in the crystallographic and powder X-ray diffraction arts, shifts in peak positions, or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art will be able to compare the figures herein with a pattern generated of an unknown form of, in this case, ranolazine, and confirm its identity as one of the forms disclosed and claimed herein. The same holds true for other techniques that may be reported herein.

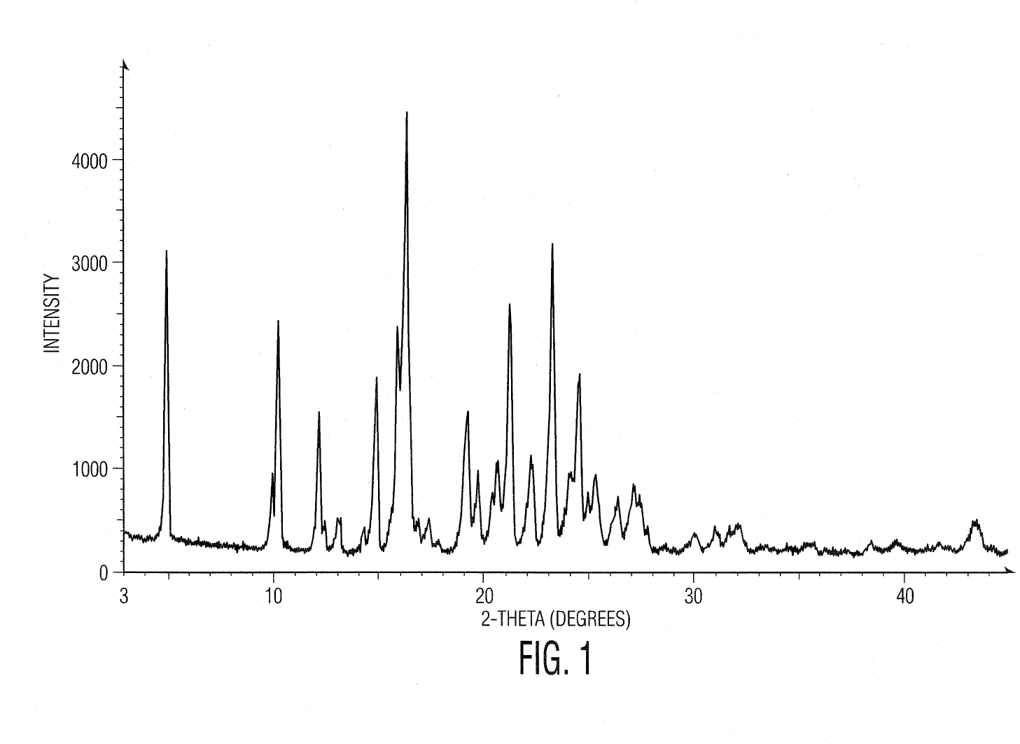

In addition, where a reference is made to a figure, it is permissible to, and this document includes and contemplates, the selection of any number of data points illustrated in the figure that uniquely define that crystalline form, salt, solvate, and/or optical isomer, within any associated and recited margin of error, for purposes of identification. Again, and as an example, for a crystalline form of ranolazine, it is permissible to select any number of PXRD peaks represented in FIG. 1 to uniquely identify that form.

Unless specified otherwise, the word “pure” means that the material has a purity at least about 99%. In general, this refers to purity with regard to unwanted residual solvents, reaction by-products, impurities, and unreacted starting materials. In the case of stereoisomers, “pure” also means 99% of one enantiomer or diastereomer, as appropriate. “Substantially pure” means purity at least about 98% and, likewise, “essentially pure” means purity at least about 95%.

The phrase, “substantially free,” as used herein in connection with impurities, unless otherwise defined, means comprising less than about 7%, or less than about 5%, or less than about 3%, or less than about 2%, or less than about 1%, or less than about 0.5%, or less than about 0.3%, or less than about 0.1%, or less than about 0.05%, by weight, of one or more of the corresponding impurities as measured using techniques such as high performance liquid chromatography (HPLC) or gas chromatography (GC).

In an aspect, the present application provides processes for the preparation of ranolazine of Formula (I) or a pharmaceutically acceptable salt thereof, embodiments of which include one or more of the following steps, individually or in the sequence recited:

(a) reacting 2-methoxyphenol with epichlorohydrin in the presence of a base, to provide 1-(2-methoxyphenoxy)-2,3-epoxypropane of Formula (II),

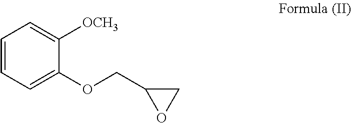

wherein the base is added to the reaction mixture in more than one portion;

(b) reacting 2,6-dimethylaniline with chloroacetylchloride in the presence of a base, to provide [(2,6-dimethylphenyl)aminocarbonylmethyl]-chloride of Formula (III);

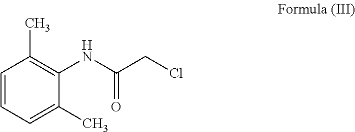

(c) reacting [(2,6-dimethylphenyl)aminocarbonylmethyl]-chloride of Formula (III) with piperazine, in a solvent, to provide N-(2,6-dimethylphenyl)-1-piperazine acetamide of Formula (IV);

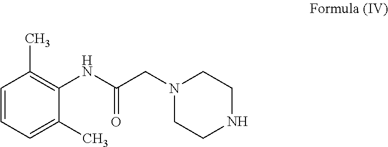

(d) reacting 1-(2-methomphenoxy)-2,3-epoxypropane of Formula (II) with N-(2,6-dimethylphenyl)-1-piperazine acetamide of Formula (IV), to provide ranolazine of Formula (I); and

(e) isolating ranolazine of Formula (I) in solid form from the reaction mixture that is obtained in (d).

Step (a) involves reacting 2-methoxy phenol with epichlorohydrin, in the presence of a base, to provide 1-(2-methoxyphenoxy)-2,3-epoxypropane of Formula (II), wherein the base is added to the reaction mixture in more than one portion.

Suitable bases that may be used in (a) include, but are not limited to: organic bases, such as, for example, triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, and the like; inorganic bases, such as, for example, alkali metal hydrides, such as, for example, sodium hydride, potassium hydride, and the like; sodamide; n-butyl lithium; lithium diisopropylamide; alkali metal hydroxides, such as, for example, lithium hydroxide, sodium hydroxide, potassium hydroxide, and cesium hydroxide; alkaline metal hydroxides, such as, for example, aluminum hydroxide, magnesium hydroxide, calcium hydroxide, and the like; alkali metal carbonates, such as, for example, sodium carbonate, potassium carbonate, lithium carbonate, cesium carbonate, and the like; alkaline earth metal carbonates, such as, for example, magnesium carbonate, calcium carbonate, and the like; alkali metal bicarbonates, such as, for example, sodium bicarbonate, potassium bicarbonate, and the like; and ion exchange resins including resins bound to ions, such as, for example, sodium, potassium, lithium, calcium, magnesium, substituted or unsubstituted ammonium, and the like; and any other suitable bases.

The present inventors have discovered that the addition of a base in small portions, at intervals of time over the entire course of the reaction, limits formation of the dimer impurity of Formula (IIa) to a level less than about 0.5%, as measured by HPLC, and a significantly improves the yield. In general, about 0.5 moles of base, or less, per mole of 2-ethoxyphenol, can be added and, after this material has reacted, the remaining quantity of base is added, in portions, and allowed to react. The base can be added in any number of portions, as long as no portion constitutes more than about 0.5 moles of base, per mole of the starting 2-ethoxyphenol. If one mole or less of base is used, per mole of 2-ethoxyphenol, an embodiment includes adding the base is two substantially equal portions.

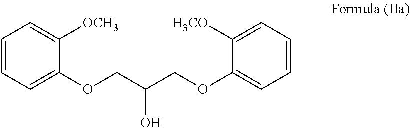

The quantities of base that may be used in (a) may be less than about 10 molar equivalents, or less than about 8 molar equivalents, or less than about 6 molar equivalents, or less than about 5 molar equivalents, or less than about 3 molar equivalents, or less than about 1 molar equivalent, or less than about 0.05 molar equivalents, or any other suitable quantity with respect to the moles of 2-methoxyphenol.

The quantity of epichlorohydrin that may be used in (a) may be less than about 10 molar equivalents, or less than about 8 molar equivalents, or less than about 7 molar equivalents, or less than about 5 molar equivalents, or less than about 3 molar equivalents, or less than about 1 molar equivalents, or less than about 0.05 molar equivalents, or any other suitable quantity with respect to the moles of 2-methoxyphenol.

Step (a) may be carried out in a suitable solvent. Suitable solvents that may be used include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxyethanol, 2-ethoxyethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitromethane; and any mixtures thereof.

Suitable temperatures for the reaction of (a) may be less than about 150° C., or less than about 100° C., or less than about 80° C., or less than about 60° C., or less than about 40° C., or less than about 30° C., or less than about 20° C., or less than about 10° C., or any other suitable temperatures.

As determined using HPLC, 1-(2-methoxyphenoxy)-2,3-epoxypropane of Formula (II) obtained according to a process of the present application may be substantially free of one or more of its corresponding impurities, e.g., the dimer impurity of Formula (IIa), the chloro impurity of Formula (IIb), and the dihydroxy impurity of Formula (IIc). For example, each of the impurities in the 1-(2-methoxyphenoxy)-2,3-epoxypropane of Formula (II) obtained according to the processes of the present invention may be present in an amount less than about 7%, or less than about 5%, or less than about 3%, or less than about 2%, or less than about 1%, or less than about 0.5%, or less than about 0.3%, or less than about 0.1%, or less than about 0.05%, by weight.

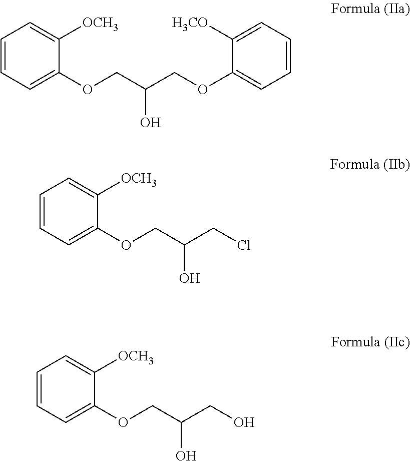

The reaction mixture containing 1-(2-methoxyphenoxy)-2,3-epoxypropane of Formula (II) obtained in (a), before or after conventional work-up, may be carried forward to (b) without first isolating the product.

Step (b) involves reacting 2,6-dimethylaniline with chloroacetylchloride, in the presence of a base, to provide [(2,6-dimethylphenyl)aminocarbonylmethyl]chloride of Formula (III).

Suitable bases that may be used in (a) include, but are not limited to: organic bases, such as, for example, triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, and the like; inorganic bases, such as, for example, alkali metal hydrides, such as, for example, sodium hydride, potassium hydride, and the like; sodamide; n-butyl lithium; lithium diisopropylamide; alkali metal hydroxides, such as, for example, lithium hydroxide, sodium hydroxide, potassium hydroxide, and cesium hydroxide; alkaline metal hydroxides, such as, for example, aluminum hydroxide, magnesium hydroxide, calcium hydroxide, and the like; alkali metal carbonates, such as, for example, sodium carbonate, potassium carbonate, lithium carbonate, cesium carbonate, and the like; alkaline earth metal carbonates, such as, for example, magnesium carbonate, calcium carbonate, and the like; alkali metal bicarbonates, such as, for example, sodium bicarbonate, potassium bicarbonate, and the like; and ion exchange resins including resins bound to ions, such as, for example, sodium, potassium, lithium, calcium, magnesium, substituted or unsubstituted ammonium, and the like; and any other suitable bases

The quantities of base that may be used in (b) may be less than about 10 molar equivalents, or less than about 8 molar equivalents, or less than about 6 molar equivalents, or less than about 5 molar equivalents, or less than about 3 molar equivalents, or less than about 1 molar equivalents, or less than about 0.05 molar equivalents, or any other suitable quantities, with respect to the moles of 2,6-dimethylaniline.

The quantities of chloroacetylchloride that may be used in (b) may be less than about 10 molar equivalents, or less than about 8 molar equivalents, or less than about 6 molar equivalents, or less than about 5 molar equivalents, or less than about 3 molar equivalents, or less than about 1 molar equivalents, or less than about 0.05 molar equivalents, or any other suitable quantities, with respect to the moles of 2,6-dimethylaniline.

Step (b) may be carried out in a suitable solvent. Suitable solvents that may be used include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxy ethanol, 2-ethoxy ethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitromethane; and any mixtures thereof.

Suitable temperatures for the reaction of (b) may be less than about 150° C., or less than about 100° C., or less than about 60° C., or less than about 40° C., or less than about 20° C., or less than about 10° C., or less than about 5° C., or less than about −10° C., or any other suitable temperatures.

The reaction mixture comprising 2-chloro-N-(2,6-dimethylphenyl) acetamide of Formula (III) obtained in (b), before or after conventional work-up, may be carried forward to (c) without first isolating the product.

2-chloro-N-(2,6-dimethylphenyl) acetamide of Formula (III) obtained in (b) may optionally be further purified until essentially pure, substantially pure, or pure. For example, 2-chloro-N-(2,6-dimethylphenyl) acetamide of Formula (III) obtained in (b) may be further purified until its purity is greater than about 99%, greater than about 99.5%, or greater than about 99.7%.

2-chloro-N-(2,6-dimethylphenyl) acetamide of Formula (III) obtained according to the processes of the present invention may be substantially free of one or more of its corresponding impurities, e.g., the dichloroacetyl impurity of Formula IIIa. For example, each of the impurities in the 2-chloro-N-(2,6-dimethylphenyl) acetamide of Formula (III) obtained according to the processes of the present invention may be present in an amount less than about 3%, or less than about 2%, or less than about 1%, or less than about 0.5%, or less than about 0.1%, or less than about 0.05%, by weight.

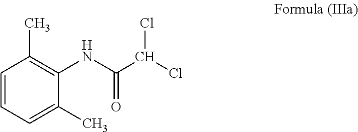

Step (c) involves reacting [(2,6-dimethylphenyl)aminocarbonylmethyl]-chloride of Formula (III) with piperazine to provide N-(2,6-dimethylphenyl)-1-piperazine acetamide of Formula (IV).

The quantities of piperazine that may be used in (c) may be less than about 6 molar equivalents, or less than about 4 molar equivalents, or less than about 3 molar equivalents, or less than about 2 molar equivalents, or less than about 1 molar equivalent, or any other suitable quantities.

Step (c) may be carried out in a suitable solvent. Suitable solvents that may be used in (c) include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxyethanol, 2-ethoxyethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitromethane; and mixtures thereof.

Suitable temperatures for the reaction of (c) may be less than about 150° C., or less than about 100° C., or less than about 60° C., or less than about 40° C., or less than about 20° C., or less than about 10° C., or less than about 5° C., or less than about −10° C., or any other suitable temperatures.

Optionally, (c) may further involve removal of a dimer impurity of Formula (IVa) by filtering the reaction mass through a medium such as diatomaceous earth.

Optionally, (c) further involves removal of unreacted piperazine by converting it into its salt form. The content of piperazine used in (c) plays a role in the formation of a dimer impurity of Formula (IVa).

The reaction mixture comprising N-(2,6-dimethylphenyl)-1-piperazine acetamide of Formula (IV) obtained in (c), before or after conventional work-up, may be carried forward to (d) without first isolating the product.

N-(2,6-dimethylphenyl)-1-piperazine acetamide of Formula (IV) obtained according to the processes of the present invention may be substantially free of one or more of its corresponding impurities, e.g., the dimer impurity of Formula (IVa). For example, each of the impurities in the N-(2,6-dimethylphenyl)-1-piperazine acetamide of Formula (IV) obtained according to the processes of the present invention may be present in an amount less than about 7%, or less than about 5%, or less than about 3%, or less than about 2%, or less than about 1%, or less than about 0.5%, as determined using HPLC.

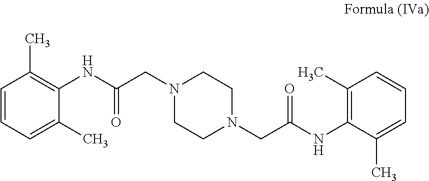

A high performance liquid chromatography method for the analysis of the dimer impurity of Formula (IVa) utilizes a C18 or equivalent column. Additional parameters are as shown in Table 1.

TABLE 1

| Flow | 0.8 mL/minute | | | | | | | |
|---|---|---|---|---|---|---|---|---|
| Detector | 210 nm | | | | | | | |
| Injection volume | 10 μL | | | | | | | |
| Temperature | 35° C. | | | | | | | |
| Mobile phase | Buffer: about 1.36 g of potassium dihydrogen | | | | | | | |
| preparation | orthophosphate and 1.5 g of n-hexanesulphonic | | | | | | | |
| | acid sodium salt in 1000 mL of purified water, | | | | | | | |
| | pH adjusted to 3.0 with dilute H₃PO₄ (1.0 mL | | | | | | | |
| | in 10 mL water). | | | | | | | |
| | Eluent A: buffer-acetonitrile | | | | | | | |
| | (90:10 by volume). | | | | | | | |
| | Eluent B: acetonitrile-methanol-water | | | | | | | |
| | (60:20:20 by volume). | | | | | | | |
| Elution | Gradient | | | | | | | |
| Gradient | Time | 0.01 | 8 | 20 | 40 | 55 | 57 | 65 |
| programme | (minute) | | | | | | | |
| | % mobile | 75 | 75 | 60 | 25 | 25 | 75 | 75 |
| | phase A | | | | | | | |
| | % of | 25 | 25 | 40 | 75 | 75 | 25 | 25 |
| | mobile | | | | | | | |
| | phase B | | | | | | | |

| | |
|---|---|
| Diluent | Eluent A. |
| Sample | 0.4 mg/mL |
| concentration | |

Step (d) involves reacting 1-(2-methoxyphenoxy)-2,3-epoxypropane of Formula (II) with N-(2,6-dimethylphenyl)-1-piperazine acetamide of Formula (IV), to provide ranolazine of Formula (I).

Step (d) may be carried out in a suitable solvent. Suitable solvents that may be used include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxyethanol, 2-ethoxyethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitromethane; and mixtures thereof.

Suitable temperatures that may be used in (d) may be less than about 150° C., or less than about 100° C., or less than about 80° C., or less than about 60° C., or less than about 40° C., or less than about 20° C., or any other suitable temperatures.

Optionally, a reaction of 1-(2-methoxyphenoxy)-2,3-epoxypropane of Formula (II) with N-(2,6-dimethylphenyl)-1-piperazine acetamide may be carried out without using a solvent medium.

Step (e) involves isolating ranolazine of Formula (I) in solid form from the reaction mixture that is obtained in step (d).

The isolation may be effected by methods including removal of solvent, cooling, concentrating the reaction mass, adding an anti-solvent, adding seed crystals, and the like. Suitable temperatures for isolation may be less than about 100° C., or less than about 60° C., or less than about 40° C., or less than about 20° C., or less than about 5° C., or less than about 0° C., or less than about −10° C., or less than about −20° C., or any other suitable temperatures. Suitable times for isolation may be less than about 5 hours, or less than about 3 hours, or less than about 2 hours, or less than about 1 hour, or longer times may be used. The exact temperatures and times required for complete isolation may be readily determined by a person skilled in the art and will also depend on parameters, such as, for example, concentrations and temperatures of the solution or slurry. Stirring or other alternate methods, such as, for example, shaking, agitation, and the like, that mix the contents may also be employed for isolation.

Suitable techniques that may be used for a removal of solvent include, but are not limited to rotational distillation using a device, such as, for example, a Buchi Rotavapor, spray drying, agitated thin-film drying, freeze drying (lyophilization), and the like, optionally under reduced pressure.

The isolated compound of Formula (I) may be recovered by methods including decantation, centrifugation, gravity filtration, suction filtration, or any other techniques for the recovery of solids. The ranolazine of Formula (I) thus isolated may carry some amount of occluded mother liquor and have higher than desired levels of impurities. The solid may be washed with a suitable solvent or a mixture of solvents, such as, for example, those used in (a), to wash out the impurities.

The isolated compound of Formula (I) may be further purified by recrystallizing one or more times from a suitable solvent or a mixture of solvents, such as, for example: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; nitromethane; and any mixtures thereof, to provide ranolazine of Formula (I) having a purity by HPLC, which is essentially pure, substantially pure, or even pure. For example, the isolated ranolazine has a purity greater than about 99%, or greater than about 99.5%, or greater than about 99.8%, or greater than about 99.9%, by weight.

The recovered solid may be optionally further dried. Drying may be carried out using a tray dryer, vacuum oven, air oven, fluidized bed dryer, spin flash dryer, flash dryer, and the like. The drying may be carried out at temperatures less than about 150° C., or less than about 120° C., or less than about 100° C., or less than about 80° C., or less than about 60° C., or any other suitable temperatures as long as the ranolazine of Formula (I) is not degraded in quality, at atmospheric pressure or under a reduced pressure. The drying may be carried out for any desired times until the required purity is achieved. For example, it may vary from about 1 to about 8 hours, or longer.

The dried product may be optionally milled to get desired particle size parameters. Milling or micronization may be performed before drying, or after the completion of drying of the product. Techniques that may be used for particle size reduction include; without limitation; sifting; milling using mills, such as, for example, ball, roller and hammer mills, and jet mills, such as, for example, air jet mill, or any other conventional techniques.

The pressures that may be used for milling or micronization are less than about 20 kg/cm², less than about 10 kg/cm², less than about 8 kg/cm², less than about 6 kg/cm², less than about 4 kg/cm², or less than about 3 kg/cm². The pressure that is applied in the mill plays an important role in reduction of particle size. The more the pressure applied, the more the reduction in particle size. By appropriately adjusting the pressure in the mill, any desired reduction in particle size may be achieved. Generally nitrogen, air or any other suitable gas may be used for applying pressure in the mill depending on the characteristics of the material going to be milled. In some cases, an inert gas such as nitrogen may have to be used in the mill to apply pressure in order to avoid formation of unwanted impurities.

In most of the particle size reduction mills, the feed rate into the mill is also an important factor in achieving reduction in particle sizes. Since the reduction in particle sizes is largely dependent on residence time of the material in the milling device, the feeder is an important device in controlling the residence time of the material in the mill, and subsequently achieving the reduction in particle sizes. It is generally observed that the higher the feed rate of the material, the lesser the reduction in particle sizes, which results in slightly coarser particle sizes of the material. On the other hand, the lower the feed rate of the material, the more the particle size reduction, which results in finer particle sizes of the material. By adjusting the appropriate feed rate, desired particle sizes may be achieved.

The desired particle sizes may also be achieved directly from the reaction mixture by selecting equipment that is able to provide ranolazine with the desired particle sizes.

In an embodiment, the present application provides processes for the preparation of N-(2,6-dimethylphenyl)-1-piperazine acetamide of Formula (IV) substantially free of piperazine, which includes one or more of the following steps:

(i) providing a mixture containing N-(2,6-dimethylphenyl)-1-piperazine acetamide of Formula (IV) in a solvent;

(ii) adjusting pH to less than about 7 with an acid; and

(iii) adjusting pH to greater than about 8 with a base and isolating N-(2,6-dimethylphenyl)-1-piperazine acetamide of Formula (IV), substantially free of piperazine.

Step (i) involves providing a mixture containing N-(2,6-dimethylphenyl)-1-piperazine acetamide of Formula (IV) in a solvent.

The mixture containing N-(2,6-dimethylphenyl)-1-piperazine acetamide of Formula (IV) in a solvent in (i) may be obtained directly from a reaction mixture containing the compound of Formula (IV).

Alternatively, the mixture containing a compound of Formula (IV) in a solvent may be obtained by combining a compound of Formula (IV) with a solvent.

Suitable solvents that may be used in (i) include, but are not limited to, water miscible solvents, including: alcohols, such as, for example, methanol, ethanol, 1-propanol, and the like; ketones, such as, for example, acetone and the like; ethers, such as, for example, tetrahydrofuran, 1,4-dioxane, and the like; nitriles, such as, for example, acetonitrile and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; water; and mixtures thereof.

Step (ii) involves adjusting pH with an acid.

Suitable acids that may be used in (ii) include, but are not limited to: organic acids, such as, for example, formic acid, acetic acid, trifluoroacetic acid, chloroacetic acid, propionic acid, butanoic acid, isobutyric acid, valeric acid, isovaleric acid, benzoic acid, salicylic acid, phthalic acid, p-toluenesulphonic acid, o-toluenesulphonic acid, benzenesulphonic acid, methanesulphonic acid, ethanesulphonic acid, phosphoric acid, sulphuric acid, and the like; ion exchange resins; chelating resins; neutral resins; or any other reagent that will bring the pH in to the desired level without affecting the quality of compound of Formula (IV). The pH of the reaction mass may be adjusted to less than about 7 or less than about 6 or less than about 5 or less than about 4 or less than about 3, or any other suitably acidic pH values.

Optionally, (ii) may be accompanied by precipitation of unreacted piperazine in the form of a salt. The precipitated piperazine in the form of a salt may be removed by methods such as, for example, decantation, centrifugation, gravity filtration, suction filtration, or any other techniques for the removal of solids.

Suitable temperatures for (ii) may be less than about 80° C., or less than about 60° C., or less than about 40° C., or less than about 20° C., or less than about 0° C., or less than about −20° C., or any other suitable temperatures.

Step (iii) involves adjusting pH with a base and isolating the N-(2,6-dimethylphenyl)-1-piperazine acetamide of Formula (IV) substantially free of piperazine.

Suitable bases that may be used in (iii) include, but are not limited to: inorganic bases, such as, for example, alkali metal hydroxides, such as, for example, lithium hydroxide, sodium hydroxide, potassium hydroxide, and cesium hydroxide; alkaline metal hydroxides, such as, for example, aluminum hydroxide, magnesium hydroxide, calcium hydroxide, and the like; alkali metal carbonates, such as, for example, sodium carbonate, potassium carbonate, lithium carbonate, cesium carbonate, and the like, alkaline earth metal carbonates, such as, for example, magnesium carbonate, calcium carbonate, and the like; alkali metal bicarbonates, such as, for example, sodium bicarbonate, potassium bicarbonate, and the like; and any other suitable bases. The pH of the reaction mass may be adjusted to greater than about 8 or greater than about 9 or greater than about 10 or greater than about 11 or greater than about 12, or any other suitably pH values.

Isolation of N-(2,6-dimethylphenyl)-1-piperazine acetamide of Formula (IV) substantially free of piperazine in (iii) may involve methods including removal of solvent, cooling, concentrating the reaction mass, adding an anti-solvent, extraction with a solvent, and the like. Stirring or other alternate methods, such as, for example, shaking, agitation, and the like, that mix the contents may also be employed for isolation.

For example, isolation of N-(2,6-dimethylphenyl)-1-piperazine acetamide of Formula (IV) substantially free of piperazine may involve extraction with a solvent. Suitable solvents that may be used for isolation of N-(2,6-dimethylphenyl)-1-piperazine acetamide of Formula (IV) include, but are not limited to: ketones, such as, for example, methyl isobutyl ketone and the like; esters, such as, for example, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, butyl methyl ether, dibutyl ether, 1,2-dimethoxyethane, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitromethane; and any mixtures thereof.

The compound of Formula (IV) may be recovered by methods including decantation, centrifugation, gravity filtration, suction filtration or any other technique for the recovery of solids. The compound of Formula (IV) thus isolated may carry some amount of occluded mother liquor and may have higher than desired levels of impurities. If desired, the solid may be washed with a solvent or a mixture of solvents to wash out the impurities.

The recovered solid may be optionally further dried. Drying may be carried out in a tray dryer, vacuum oven, air oven, fluidized bed dryer, spin flash dryer, flash dryer, and the like. The drying may be carried out at temperatures less than about 150° C., or less than about 120° C., or less than about 100° C., or less than about 80° C., or less than about 60° C., or any other suitable temperatures as long as the compound of Formula (IV) is not degraded in quality, at atmospheric pressure or under a reduced pressure. The drying may be carried out for any desired times until the required purity is achieved. For example, it may vary from about 1 to about 10 hours, or longer.

In an embodiment, the present application provides N-(2,6-dimethylphenyl)-1-piperazine acetamide of Formula (IV) substantially free of piperazine.

The present inventors have discovered that the content of piperazine in the N-(2,6-dimethylphenyl)-1-piperazine acetamide of Formula (IV) plays a role in the formation of the dimer impurity of Formula (Va), in processes for preparing ranolazine. For example, the present inventors have discovered that N-(2,6-dimethylphenyl)-1-piperazine acetamide of Formula (IV) having a content of piperazine greater than about 0.1% leads to the formation of the dimer impurity of Formula (Va) in ranolazine at a undesired level. In a large scale operation, it is very difficult to purify ranolazine having such an undesired level of the dimer impurity of Formula (Va) using conventional techniques.

The phrase, “substantially free of piperazine,” as used herein, means the compound contains less than about 1%, or less than about 0.5%, or less than about 0.1%, or less than about 0.05%, or less than about 0.01%, or less than about 0.008%, or less than about 0.005%, by weight of piperazine as measured by gas chromatography.

A gas chromatography method used for the analysis of piperazine utilizes an AT-1701 or equivalent column. Additional method parameters are as shown in Table 2.

TABLE 2

| | |
|---|---|
| Length | 30 meters |
| ID | 0.53 mm |
| Film thickness | 1.2 μm |
| Detector | 260° C. |
| Injector | 250° C. |
| Split ratio | 1:5 |
| Carrier gas | Helium, 2.5 psi (24 kPa) |
| Load | 1.0 μL |
| Diluent | Methanol |
| Sample | 100 mg/mL in methanol |
| concentration | |
| Column oven | Initially held at 150° C. for 0 minutes, then |
| temperature | increased to 270° C. at a rate of 25° C. per minute |
| | and held at 270° C. for 20 minutes. |

Two crystalline polymorphic forms of N-(2,6-dimethylphenyl)-1-piperazine acetamide of Formula (IV) have been encountered, and these will be described as “Form A” and “Form B.”

Form A of N-(2,6-dimethylphenyl)-1-piperazine acetamide of Formula (IV) prepared according to a process of the present application may be characterized by a powder X-ray diffraction pattern having peak locations substantially as listed in Table 3.

TABLE 3

| 2θ (degrees) ± 0.2 | d-spacing (Å) ± 0.02 |
|---|---|
| 10.7 | 8.20 |
| 13.1 | 6.71 |
| 13.3 | 6.60 |
| 14.5 | 6.06 |
| 16.0 | 5.50 |
| 16.5 | 5.35 |
| 16.8 | 5.25 |
| 17.8 | 4.96 |
| 18.3 | 4.82 |
| 18.8 | 4.69 |
| 19.5 | 4.53 |
| 20.1 | 4.41 |
| 20.8 | 4.26 |
| 21.6 | 4.10 |
| 22.7 | 3.89 |
| 23.1 | 3.84 |
| 24.4 | 3.63 |
| 24.7 | 3.59 |
| 26.4 | 3.36 |
| 27.7 | 3.21 |
| 28.1 | 3.17 |
| 30.0 | 2.96 |
| 32.5 | 2.74 |
| 33.9 | 2.63 |
| 34.3 | 2.60 |
| 38.1 | 2.35 |
| 39.9 | 2.25 |

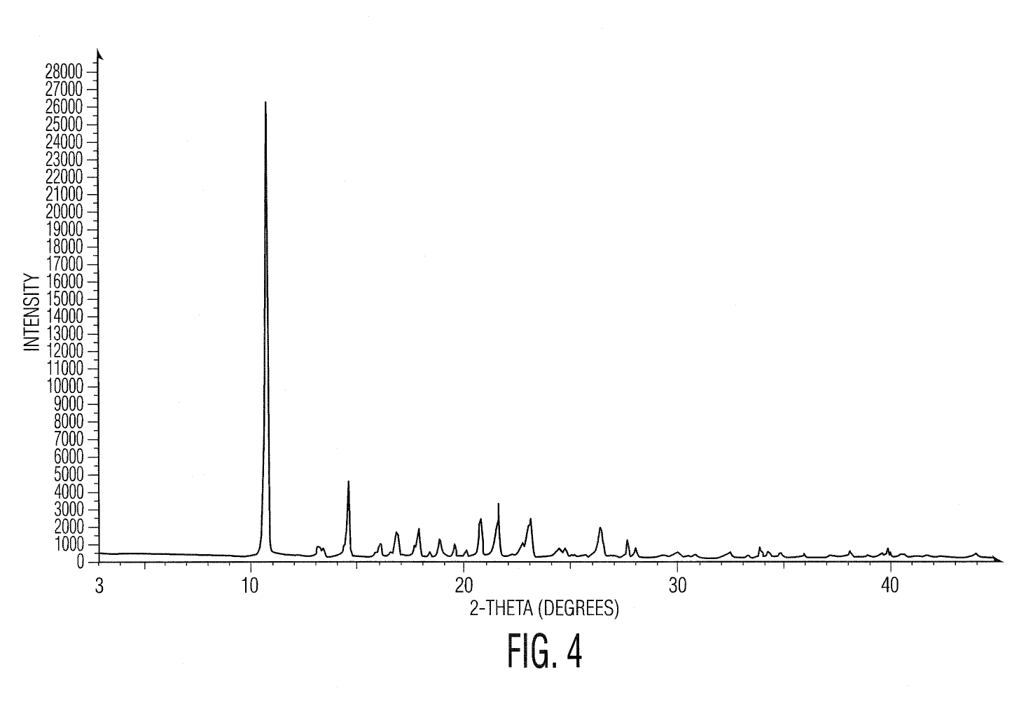

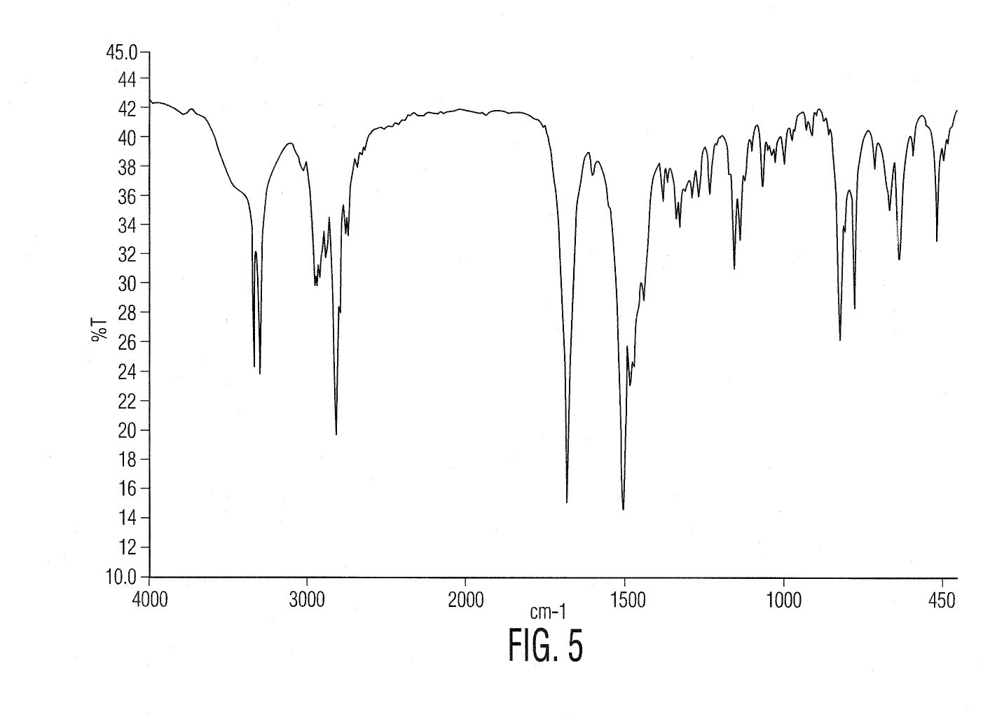

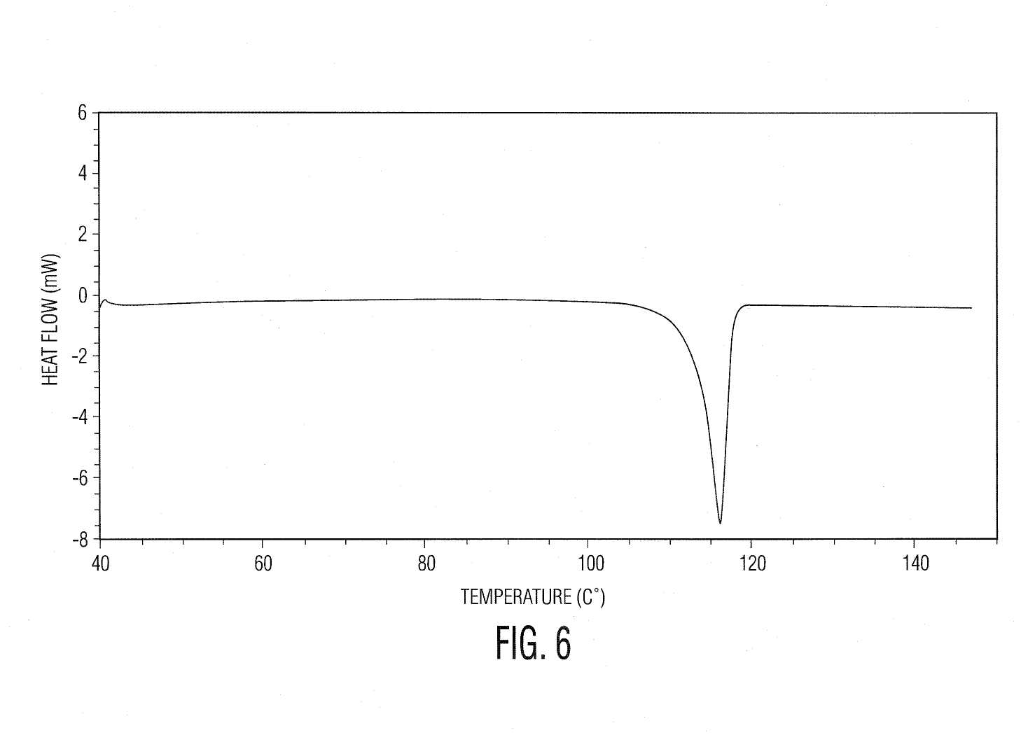

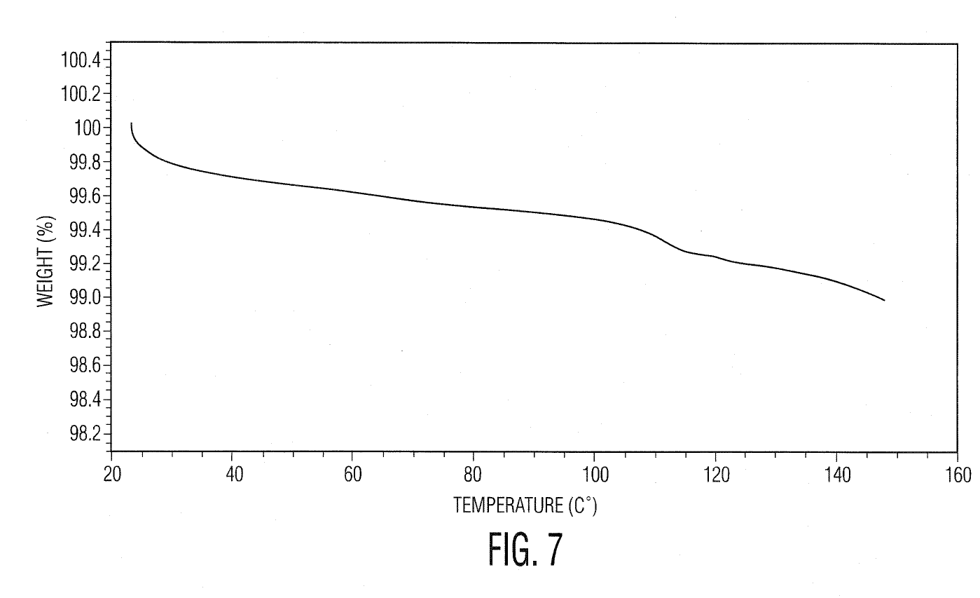

Form A of N-(2,6-dimethylphenyl)-1-piperazine acetamide of Formula (IV) prepared according to a process of the present application may be characterized by any one or more of a powder X-ray diffraction pattern, infrared absorption spectrum, differential scanning calorimetry (DSC) thermogram and thermogravimetric analysis (TGA) curve that, respectively, may be substantially as illustrated by FIGS. 4, 5, 6, and 7.

Form B of N-(2,6-dimethylphenyl)-1-piperazine acetamide of Formula (IV) prepared according to a process of the present application may be characterized by a powder X-ray diffraction pattern having peak locations substantially as listed in Table 4.

TABLE 4

| 2θ (degrees) ± 0.2 | d-spacing (Å) ± 0.02 |
|---|---|
| 6.3 | 13.89 |
| 7.9 | 11.05 |
| 8.6 | 10.24 |
| 12.7 | 6.91 |
| 13.4 | 6.56 |
| 14.3 | 6.15 |
| 15.1 | 5.86 |
| 16.0 | 5.52 |
| 16.7 | 5.30 |
| 19.0 | 4.65 |
| 19.7 | 4.48 |
| 20.5 | 4.30 |
| 21.3 | 4.16 |
| 21.9 | 4.04 |
| 22.8 | 3.89 |
| 23.1 | 3.84 |
| 24.0 | 3.69 |
| 24.5 | 3.62 |
| 25.4 | 3.49 |
| 26.8 | 3.31 |
| 27.0 | 3.29 |
| 27.4 | 3.25 |
| 28.2 | 3.15 |
| 32.4 | 2.75 |
| 32.9 | 2.71 |
| 34.5 | 2.59 |
| 35.3 | 2.53 |
| 37.2 | 2.41 |
| 40.8 | 2.21 |
| 42.6 | 2.12 |

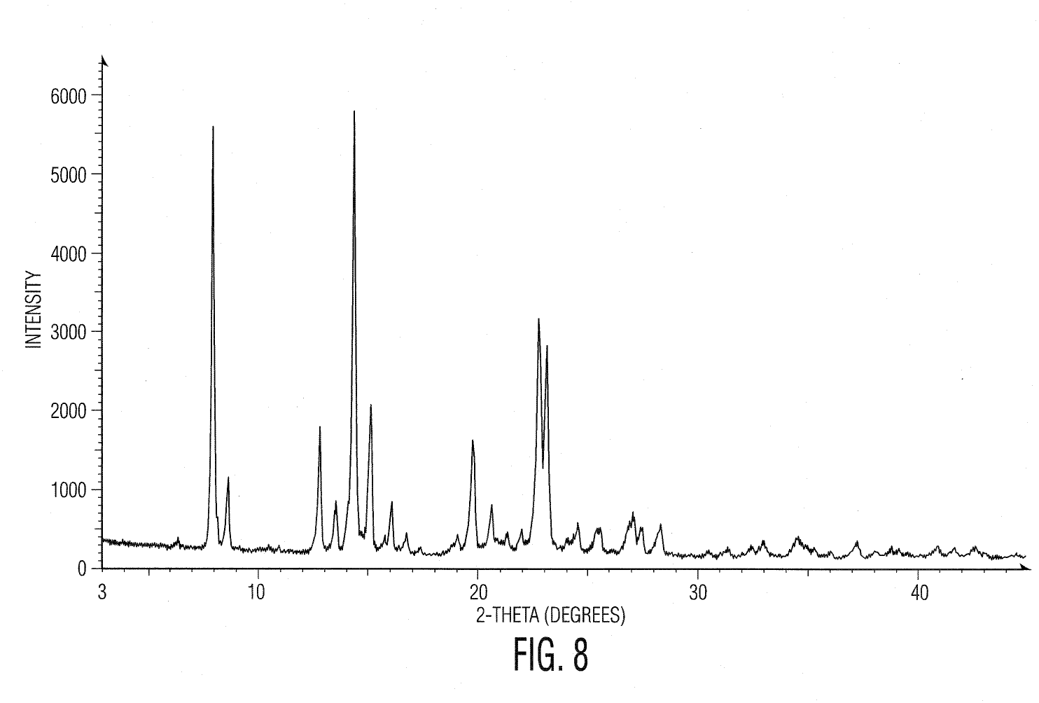

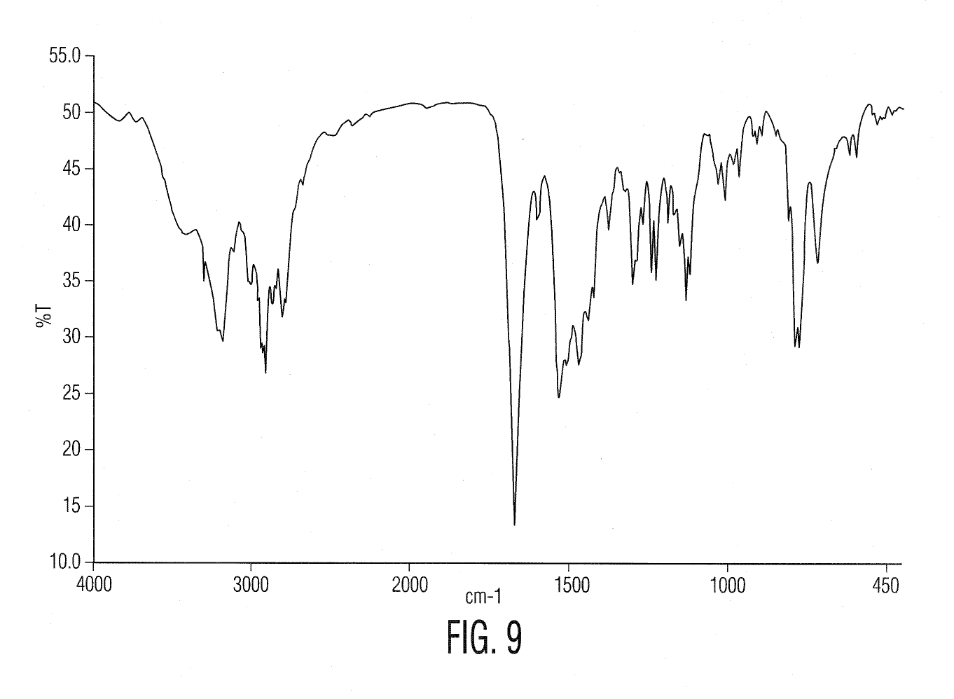

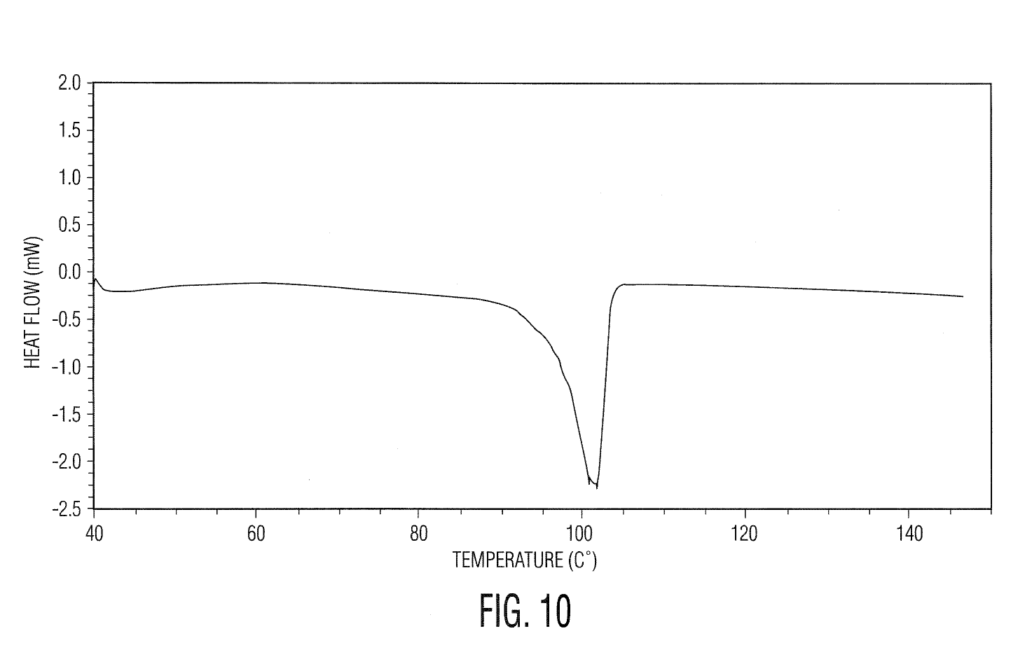

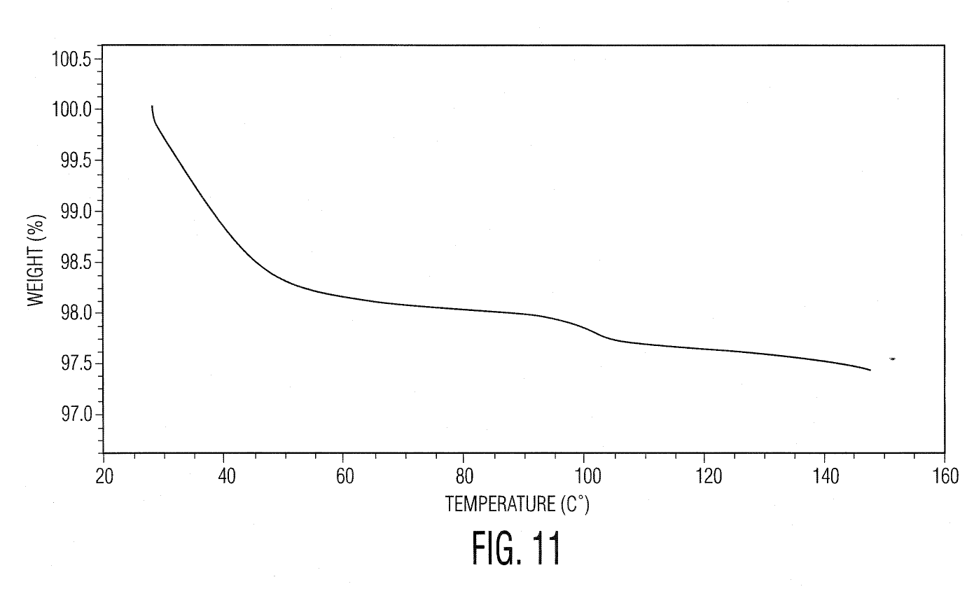

Form B of N-(2,6-dimethylphenyl)-1-piperazine acetamide of Formula (IV) prepared according to a process of the present application may be characterized by any one or more of a powder X-ray diffraction pattern, infrared absorption spectrum, differential scanning calorimetry (DSC) thermogram, and thermogravimetric analysis (TGA) curve that, respectively, may be substantially as illustrated by FIGS. 8, 9, 10, and 11.

All PXRD data reported herein were obtained using a Bruker AXS D8 Advance Powder X-ray Diffractometer with copper Kα radiation.

Differential scanning calorimetric analyses reported herein were carried out using a DSC Q1000 model from TA Instruments with a ramp of 10° C./minute up to 150° C. The starting temperature was 40° C. and ending temperature was 150° C.

Thermogravimetric analysis analyses reported herein were carried out using a TGA Q500 V6.4 Build 193 from TA Instruments, with a ramp of 5° C./minute up to 150° C.

In embodiments, the present application provides processes for the preparation of ranolazine of Formula (I) or a pharmaceutically acceptable salt thereof, which include one or more of the following steps, individually or in the sequence recited:

(a) reacting 2-methoxyphenol with epichlorohydrin in the presence of a base, to provide 1-(2-methoxyphenoxy)-2,3-epoxypropane of Formula (II),

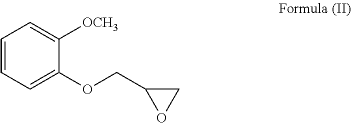

wherein the base is added to the reaction mixture in small portions.

(b) reacting 1-(2-methoxyphenoxy)-2,3-epoxypropane of Formula (II) with piperazine in a solvent, to provide 1-[3-(2-methoxyphenoxy)-2-hydroxypropyl]-piperazine of Formula (V);

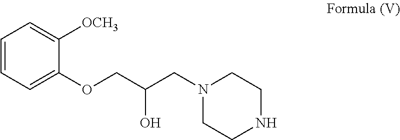

(c) reacting 1-[3-(2-methoxyphenoxy)-2-hydroxypropyl]-piperazine of Formula (V) with [(2,6-dimethylphenyl)aminocarbonylmethyl]-chloride of Formula (III) in a solvent, to provide ranolazine of Formula (I); and

(d) isolating ranolazine of Formula (I) in solid form from the reaction mixture that is obtained in step (c).

Step (a) involves reacting 2-methoxyphenol with epichlorohydrin in the presence of a base, to provide 1-(2-methoxyphenoxy)-2,3-epoxypropane of Formula (II), wherein the base is added to the reaction mixture in small portions.

Suitable bases that may be used in (a) include, but are not limited to: organic bases, such as, for example, triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, and the like; inorganic bases, such as, for example, alkali metal hydrides, such as, for example, sodium hydride, potassium hydride, and the like; sodamide; n-butyl lithium; lithium diisopropylamide; alkali metal hydroxides, such as, for example, lithium hydroxide, sodium hydroxide, potassium hydroxide, and cesium hydroxide; alkaline metal hydroxides, such as, for example, aluminum hydroxide, magnesium hydroxide, calcium hydroxide, and the like; alkali metal carbonates, such as, for example, sodium carbonate, potassium carbonate, lithium carbonate, cesium carbonate, and the like; alkaline earth metal carbonates, such as, for example, magnesium carbonate, calcium carbonate, and the like; alkali metal bicarbonates, such as, for example, sodium bicarbonate, potassium bicarbonate, and the like; ion exchange resins including resins bound to ions, such as, for example, sodium, potassium, lithium, calcium, magnesium, substituted or unsubstituted ammonium, and the like; and any other suitable bases.

The present inventors have discovered that the addition of a base in small portions limits formation of the dimer impurity of Formula (IIa) to levels less than about 0.5% as determined by HPLC, and significantly improves the yield.

The phrase, “in small portions,” as used herein, means addition of the base in divided amounts to the reaction mixture, at intervals of time over the entire course of the reaction. For example, less than about 50% of the required moles of base can be added, allowed to react, then the remaining amount of base can be added in one or more additional portions.

The quantities of base that may be used in (a) may be less than about 10 molar equivalents, or less than about 8 molar equivalents, or less than about 6 molar equivalents, or less than about 5 molar equivalents, or less than about 3 molar equivalents, or less than about 1 molar equivalent, or less than about 0.05 molar equivalents, or any other suitable quantities, with respect to the moles of 2-methoxyphenol.

The quantity of epichlorohydrin that may be used in (a) may be less than about 10 molar equivalents, or less than about 8 molar equivalents, or less than about 7 molar equivalents, or less than about 5 molar equivalents, or less than about 3 molar equivalents, or less than about 1 molar equivalent, or less than about 0.05 molar equivalents, or any other suitable quantity, with respect to the moles of 2-methoxyphenol.

Step (a) may be carried out in a suitable solvent. Suitable solvents that may be used in (a) include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxyethanol, 2-ethoxyethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitromethane; and any mixtures thereof.

Suitable temperatures that may be used for the reaction of (a) may be less than about 150° C., or less than about 100° C., or less than about 80° C., or less than about 60° C., or less than about 40° C., or less than about 30° C., or less than about 20° C., or less than about 10° C., or any other suitable temperatures. 1-(2-methoxyphenoxy)-2,3-epoxypropane of Formula (II) obtained according to the processes of the present application may be substantially free of one or more of its corresponding impurities as determined by HPLC, e.g., the dimer impurity of Formula (IIa), the chloro impurity of Formula (IIb), and the dihydroxy impurity of Formula (IIc). For example, each of the impurities may be present in an amount less than about 7%, or less than about 5%, or less than about 3%, or less than about 2%, or less than about 1%, or less than about 0.5%, or less than about 0.3%, or less than about 0.1%, or less than about 0.05%, by weight.

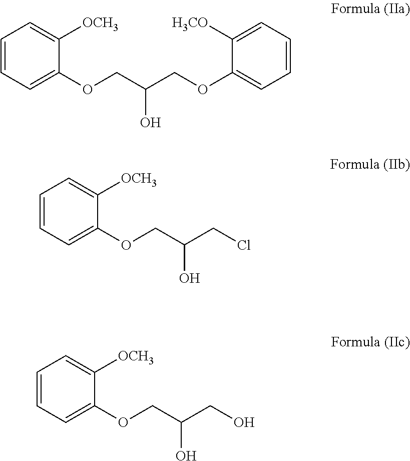

A high performance liquid chromatography method for the analysis of the dimer impurity of Formula (IIa), chloro impurity of Formula (IIb), dihydroxy impurity of (IIc), and dichloroacetyl impurity of Formula IIIa utilizes a C18 or equivalent column. Additional parameters are as shown in Table 5 and representative relative retention times (ranolazine=1) are in Table 6.

TABLE 5

| | |
|---|---|
| Flow | 1.0 mL/minute |
| Elution | gradient |
| Detector | 223 nm |
| Injection volume | 10 μL |
| Oven temperature | ambient |
| Mobile phase | Eluent A: Water pH adjusted to 5.0 with dilute H₃PO₄. |
| preparation | Eluent B: acetonitrile-water (80:20 by volume). |
| Run time | 65 minutes |
| Diluent | Acetonitrile-water (30:70 by volume) |
| Sample | 1 mg/mL |
| concentration | |

TABLE 6

| | Dimer | | Dihydroxy | Dichloroacetyl |
|---|---|---|---|---|
| | impurity of | Chloro impurity | impurity of | impurity of |
| | Formula IIa | of Formula IIb | Formula IIc | Formula IIIa |
| RRT | 1.61 | 1.14 | 0.34 | 1.54 |

The reaction mixture containing 1-(2-methoxyphenoxy)-2,3-epoxypropane of Formula (II) obtained in (a), before or after conventional work-up, may be carried forward to (b) without isolating the product.

Step (b) involves reacting 1-(2-methoxyphenoxy)-2,3-epoxypropane of Formula (II) with piperazine, to provide 1-[3-(2-methoxyphenoxy)-2-hydroxypropyl]-piperazine of Formula (V).

Step (b) may be carried out in a suitable solvent. Suitable solvents that may be used in (b) include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxyethanol, 2-ethoxyethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitro methane; and any mixtures thereof.

The reaction mixture comprising 1-[3-(2-methoxyphenoxy)-2-hydroxypropyl]-piperazine of Formula (V) obtained in (b), before or after conventional work-up, may be carried forward to (c) without isolating the product.

Step (c) involves reacting 1-[3-(2-methoxyphenoxy)-2-hydroxypropyl]-piperazine of Formula (V) with [(2,6-dimethylphenyl)aminocarbonylmethyl]-chloride of Formula (III), to provide ranolazine of Formula (I).

Step (c) may be carried out in a suitable solvent. Suitable solvents that may be used in (c) include, but are not limited to: water; alcohols, such as, for example, methanol, ethanol, propanol, butanol, pentanol, ethylene glycol, glycerol, and the like; ketones, such as, for example, acetone, butanone, pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; ethers, such as, for example, diethyl ether, diisopropyl ether, t-butyl methyl ether, dibutyl ether, tetrahydrofuran, 1,2-dimethoxyethane, 2-methoxyethanol, 2-ethoxyethanol, anisole, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; polar aprotic solvents, such as, for example, N,N-dimethylformamide, N,N-dimethylacetamide, N-methylpyrrolidone, pyridine, dimethylsulphoxide, sulpholane, formamide, acetamide, propanamide, and the like; nitromethane; and any mixtures thereof.

Suitable temperature for the reaction of (c) may be less than about 150° C., or less than about 100° C., or less than about 80° C., or less than about 60° C., or less than about 40° C., or less than about 20° C., or any other suitable temperatures.

Step (d) involves isolating ranolazine in solid form from the reaction mixture that is obtained in (c).

The isolation step may be affected by methods including removal of solvent, cooling, concentrating the reaction mass, adding an anti-solvent, adding seed crystals, and the like. Suitable temperature for isolation may be less than about 100° C., or less than about 60° C., or less than about 40° C., or less than about 20° C., or less than about 5° C., or less than about 0° C., or less than about −10° C., or less than about −20° C., or any other suitable temperatures. Suitable times for isolation may be less than about 5 hours, or less than about 3 hours, or less than about 2 hours, or less than about 1 hour, or longer times may be used. The exact temperature and time required for complete isolation may be readily determined by a person skilled in the art and will also depend on parameters, such as, for example, concentration and temperature of the solution or slurry. Stirring or other alternate methods, such as, for example, shaking, agitation, and the like, that mix the contents may also be employed for isolation.

Suitable techniques that may be used for the removal of solvent include, but are not limited to, rotational distillation using a device, such as, for example, a Buchi Rotavapor, spray drying, agitated thin-film drying, freeze drying (lyophilization), and the like, optionally under reduced pressure.

The isolated compound of Formula (I) may be recovered by methods including decantation, centrifugation, gravity filtration, suction filtration, or any other techniques for the recovery of solids. The ranolazine of Formula (I) thus isolated may carry some amount of occluded mother liquor and thus have higher than desired levels of impurities. If desired, the solid may be washed with a suitable solvent or a mixture of solvents, such as, for example, those used in (a), to wash out the impurities.

The isolated compound of Formula (I) may be further purified by recrystallization one or more times from a suitable solvent or a mixture of solvents, such as, for example: water; alcohols, such as, for example, methanol, ethanol, propanol, butanol, pentanol, ethylene glycol, glycerol, and the like; ketones, such as, for example, acetone, butanone; pentanone, methyl isobutyl ketone, and the like; esters, such as, for example, ethyl formate, methyl acetate, ethyl acetate, propyl acetate, butyl acetate, methyl propanoate, ethyl proponoate, methyl butanoate, ethyl butanoate, and the like; aliphatic or alicyclic hydrocarbons, such as, for example, hexane, heptane, pentane, cyclohexane, methylcyclohexane, and the like; halogenated hydrocarbons, such as, for example, dichloromethane, chloroform, 1,1,2-trichloroethane, 1,2-dichloroethene, and the like; aromatic hydrocarbons, such as, for example, toluene, xylene, chlorobenzene, tetraline, and the like; nitriles, such as, for example, acetonitrile, propionitrile, and the like; nitro methane; and any mixtures thereof, to provide ranolazine of Formula (I) having a purity by HPLC, which is essentially pure, substantially pure, or even pure. For example, the isolated ranolazine has a purity greater than about 99%, or greater than about 99.5%, or greater than about 99.8%, or greater than about 99.9%, by weight.

The recovered solid may be optionally further dried. Drying may be carried out in a tray dryer, vacuum oven, air oven, fluidized bed dryer, spin flash dryer, flash dryer, and the like. The drying may be carried out at temperatures less than about 150° C., or less than about 120° C., or less than about 100° C., or less than about 80° C., or less than about 60° C., or any other suitable temperatures as long as the ranolazine of Formula (I) is not degraded in quality, at atmospheric pressure or under a reduced pressure. The drying may be carried out for any desired time until the required purity is achieved. For example, it may vary from about 1 to about 8 hours, or longer.

The dried product may be optionally milled to get desired particle sizes. Milling or micronization may be performed before drying, or after the completion of drying of the product. Techniques that may be used for particle size reduction include, without limitation, sifting, milling using mills, such as, for example, ball, roller and hammer mills, and jet mills, such as, for example, air jet mill, or any other conventional techniques.

The pressures that may be used for milling or micronization typically are less than about 20 kg/cm², less than about 10 kg/cm², less than about 8 kg/cm², less than about 6 kg/cm², less than about 4 kg/cm², less than about 3 kg/cm². The pressure that is applied in the mill plays an important role in reduction of particle size. The more the pressure applied, the more the reduction in particle size. By appropriately adjusting the pressure in the mill, any desired reduction in particle size may be achieved. Generally nitrogen, air or any other suitable gas may be used for applying pressure in the mill depending on the characteristics of the material going to be milled. In some cases, an inert gas such as nitrogen may have to be used in the mill to apply pressure, in order to avoid formation of unwanted impurities.

In most of the particle size reduction mills, the feed rate into the mill is also an important factor in achieving reduction in particle sizes. Since the reduction in particle sizes is largely dependent on residence time of the material in the milling device, the feeder is an important device in controlling the residence time of the material in the mill, and subsequently achieving the reduction in particle sizes. It is generally observed that the higher the feed rate of the material, the lesser the reduction in particle sizes, which results in slightly coarser particle sizes of the material. On the other hand, the lower the feed rate of the material, the more the particle size reduction, which results in finer particle sizes of the material. By adjusting the appropriate feed rate, desired particle size may be achieved.

The desired particle sizes may also be achieved directly from the reaction mixture by selecting appropriate equipment, such as, for example, appropriate agitator and reactor, which are suitable to provide ranolazine with desired particle size.

In an embodiment, the present application provides processes for the preparation of ranolazine in a solid form, comprising at least one of the steps of:

(a) reacting the compound of Formula (II) with the compound of Formula (IV); and

(b) reacting the compound of Formula (III) with the compound for Formula (V), and in either event isolating ranolazine of Formula (I) in solid form.

In an embodiment, the present application provides processes for the preparation of ranolazine, wherein the ranolazine is isolated without having been in the form of a salt.

In an embodiment, the present application provides processes for the preparation of ranolazine, wherein the processes do not include use of high vacuum distillation or column chromatography techniques.

Ranolazine of Formula (I) obtained according to the processes of the present application may be substantially free of one or more of its corresponding impurities.

EMEA/CHMP/ICH/126642/2008 Guidance on Genotoxicity Testing and Data Interpretation for Pharmaceuticals Intended for Human Use discloses, “Certain structurally alerting molecular entities are recognized as being causally related to the carcinogenic and/or mutagenic potential of chemicals. Examples of structural alerts include alkylating electrophilic centers, unstable epoxides, aromatic amines, azo-structures, N-nitroso groups, and aromatic nitro-groups (Ashby and Paton 1994). For some classes of compounds with specific structural alerts, it is established that specific protocol modifications/additional tests are important for optimum detection of genotoxicity.”

EMEA/CHMP/QWP/251344/2006 Guideline on the limits of Genotoxic Impurities discloses, “the applicant should highlight, within the chemical process and impurity profile of active substance, all chemical substances, used as reagents or present as intermediates, or side-products, known as genotoxic and/or carcinogenic (e.g. alkylating agents). More generally, reacting substances and substances which show “alerting structure” in terms of genotoxicity which are not shared with the active substance should be considered (see e.g. Dobo et al. 2006). Potential alternatives which do not lead to genotoxic residues in the final product should be used if available.”

“Substantially free of one or more of its corresponding impurities” as used herein, unless otherwise defined refers to the compound that contains less than about 1%, or less than about 0.5%, or less than about 0.3%, or less than about 0.1%, or less than about 0.05%, or less than about 0.01%, or less than about 0.005%, or less than about 0.001%, or less than about 5 ppm, or less than about 3 ppm, or less than about 2 ppm, or less than about 1 ppm, or less than about 0.5 ppm, or less than about 0.3 ppm, or less than about 0.2 ppm, or less than about 0.1 ppm, by weight, of each individual impurity including, without limitation, the compound of Formula (II), the dimer impurity of Formula (IIa), the chloro impurity of Formula (IIb), the dihydroxy impurity of Formula (IIc), the compound of Formula (III), the dichloroacetyl impurity of Formula (IIIa), the compound of Formula (IV), the dimer impurity of Formula (IVa), the compound of Formula (V), the dimer impurity of Formula (Va), the ECH dimer impurity of Formula (VI), the impurity at the impurities at RRT (relative retention time) 0.73 having a mass number 443, the impurity at RRT 0.56 having a mass number 459, 2,6-dimethylaniline of Formula (VII), epichlorohydrin of Formula (VIII), chloroacetic acid of Formula (IX), or any other drug-related or process-related impurity, and that contains a total amount of impurities of less than about 1%, or less than about 0.5%, or less than about 0.3%, or less than about 0.1%, or less than about 0.05%, less than about 0.01% or less than about 0.005%, less than about 0.001%, or less than about 5 ppm, or less than about 3 ppm, or less than about 2 ppm, or less than about 1 ppm, or less than about 0.5 ppm, by weight.

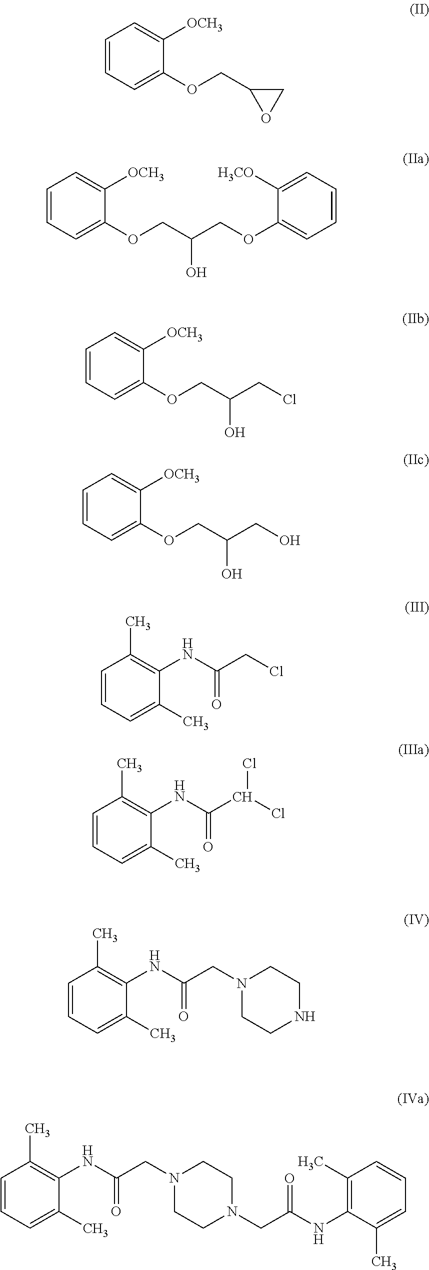

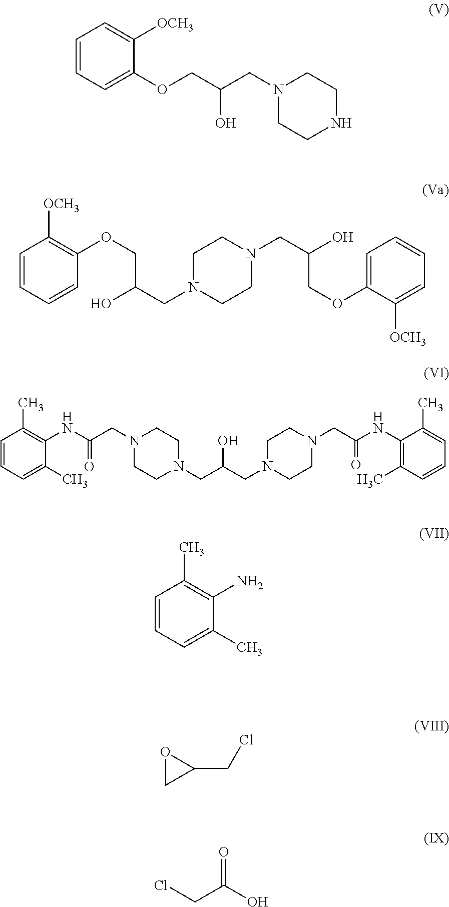

A high performance liquid chromatography method for the analysis of a compound of Formula (I) utilizes a L1 or equivalent column. Additional parameters are as shown in Table 7.

TABLE 7

| Flow | 1.0 mL/minute | | | | | | | |
|---|---|---|---|---|---|---|---|---|
| Detector | 223 nm | | | | | | | |
| Injection volume | 10 μL | | | | | | | |
| Temperature | Ambient | | | | | | | |
| Mobile phase | Buffer preparation: 1.38 g of NaH₂PO₄•H₂O in | | | | | | | |
| preparation | 1000 mL of water, add 1.0 mL of triethylamine | | | | | | | |
| | then adjust the pH to 7.3 with dilute | | | | | | | |
| | orthophosphoric acid (1.0 mL in 10 mL). | | | | | | | |
| | Mobile phase A: Degassed mixture of buffer and | | | | | | | |
| | acetonitrile in the ratio of 9:1 by volume. | | | | | | | |
| | Mobile phase B: Degassed mixture of buffer and | | | | | | | |
| | acetonitrile in the ratio of 45:55 by volume. | | | | | | | |
| Diluent | Acetonitrile-water | | | | | | | |
| Sample | 3.0 mg/mL | | | | | | | |
| concentration | | | | | | | | |
| Elution | Gradient | | | | | | | |
| Gradient | Time | 0.01 | 8 | 25 | 30 | 55 | 57 | 65 |
| program | (minute) | | | | | | | |
| | % of | 83 | 83 | 30 | 25 | 25 | 83 | 83 |
| | mobile | | | | | | | |
| | phase A | | | | | | | |
| | % of | 17 | 17 | 70 | 75 | 75 | 17 | 17 |
| | mobile | | | | | | | |
| | phase B | | | | | | | |

A high performance liquid chromatography method for the analysis of a compound of Formula (II), the compound of Formula (III), and the chloro impurity of Formula (IIb), utilizes a L1 or equivalent column. Additional parameters are as shown in Table 8.

TABLE 8

| Flow | 1.0 mL/minute | | | | | | | |
|---|---|---|---|---|---|---|---|---|
| Detector | 223 nm | | | | | | | |
| Temperature | Ambient | | | | | | | |
| Load | 220 μL | | | | | | | |
| Mobile phase | Dissolve 1.38 g of sodium dihydrogen | | | | | | | |
| preparation | phosphate monohydrate in 1000 mL of milli-Q | | | | | | | |
| | water and adjust the pH to 7.3 with dilute | | | | | | | |
| | phosphoric acid. | | | | | | | |
| | Mobile phase A: Buffer and acetonitrile in the | | | | | | | |
| | volume ratio of 9:1. | | | | | | | |
| | Mobile phase B: Buffer and acetonitrile in the | | | | | | | |
| | ratio of 450 mL to 550 mL. | | | | | | | |
| Diluent | Acetonitrile-water | | | | | | | |
| Sample | 30 mg/mL | | | | | | | |
| concentration | | | | | | | | |
| Elution | Gradient | | | | | | | |
| Gradient | Time | 0.01 | 12 | 25 | 30 | 45 | 47 | 55 |
| program | (minute) | | | | | | | |
| | % of | 65 | 65 | 30 | 20 | 20 | 65 | 65 |
| | mobile | | | | | | | |
| | phase A | | | | | | | |
| | % of | 35 | 35 | 70 | 80 | 80 | 35 | 35 |
| | mobile | | | | | | | |
| | phase B | | | | | | | |

A high performance liquid chromatography method for the analysis of the dichloroacetyl impurity of Formula (IIIa) utilizes a L1 or equivalent column. Additional parameters are as shown in Table 9.

TABLE 9

| Flow | 1.2 mL/minute | | | | | | |
|---|---|---|---|---|---|---|---|
| Detector | 223 nm | | | | | | |
| Temperature | Ambient | | | | | | |
| Load | 50 μL | | | | | | |
| Mobile phase | Preparation of buffer: Dissolve 1.36 g of | | | | | | |
| preparation | potassium dihydrogen phosphate and 0.8 g | | | | | | |
| | tetrabutylammonium hydrogen sulphate in | | | | | | |
| | 1000 mL of milli-Q water. | | | | | | |
| | Mobile phase A: Buffer. | | | | | | |
| | Mobile phase B: Water and acetonitrile in the | | | | | | |
| | volume ratio of 3:7. | | | | | | |
| Diluent | Mobile phase B | | | | | | |
| Sample | 100 mg/mL | | | | | | |
| concentration | | | | | | | |
| Elution | Gradient | | | | | | |
| Gradient | Time | 0.01 | 3 | 20 | 28 | 30 | 37 |
| program | (Minute) | | | | | | |
| | % of | 80 | 80 | 20 | 20 | 80 | 80 |
| | mobile | | | | | | |
| | phase A | | | | | | |
| | % of | 20 | 20 | 80 | 80 | 20 | 20 |
| | mobile | | | | | | |
| | phase B | | | | | | |

A gas chromatography method used for the analysis of 2,6-dimethylaniline of Formula (VII) utilizes a G43 or equivalent column. Additional parameters are as shown in Table 10.

TABLE 10

| | |
|---|---|
| Inlet pressure | 9 psi (62 kPa) |
| Injector, Detector | 210° C., 260° C. with FID |
| temperatures | |
| Injection volume | 4 μL |
| Inlet mode | Split |
| Column oven | Initially 80° C. for 4 minutes, increased to 220° |
| temperature: | C. at a rate of 20° C. per minute, then further |
| | increased to 260° C. at a rate of 80° C. per minute |
| | and held for 10 minutes. |
| Diluent | Dichloromethane-methanol |
| Sample | 130 mg/mL |
| concentration | |
| Make up flow | 30 mL/minute |
| Fuel flow | 30 mL/minute |
| Oxidizer flow | 300 mL/minute |

A gas chromatography method used for the analysis of epichlorohydrin of Formula (VIII) utilizes a G43 or equivalent column. Additional parameters are as shown in Table 11.

TABLE 11

| | |
|---|---|
| Inlet pressure | 6 psi (41 kPa) |
| Injector, Detector | 130° C., 280° C. with FID |
| temperatures | |
| Injection volume | 4 μL |
| Inlet mode | Splitless |
| Column oven | Initially held at 40° C. for 2 minutes, then increased to |
| temperature: | 240° C. at a rate of 10° C. per minute and held for 4 |
| | minutes. |
| Diluent | Dichloromethane |
| Sample | 300 mg/mL |
| concentration | |
| Make up flow | 30 mL/minute |
| Fuel flow | 30 mL/minute |
| Oxidizer flow | 300 mL/minute |

For example, ranolazine of Formula (I) prepared according to a processes of the present application may be characterized by an X-ray powder diffraction pattern having characteristic peaks at about 4.9, 9.9, 10.2, 12.1, 14.8, 15.9, 16.4, 19.2, 19.7, 21.3, 22.2, 23.3, 24.1, 24.5, 24.9, 25.3, 26.4, 27.1, and 27.4, ±0.2 degrees 2θ.

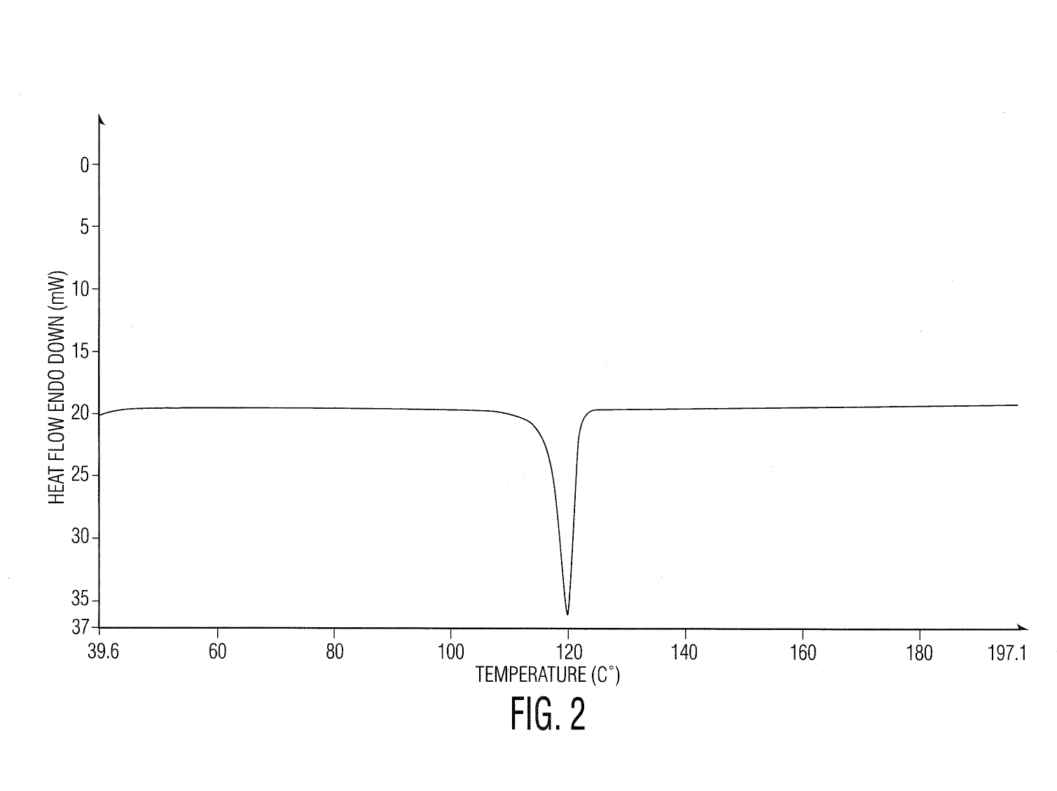

For example, ranolazine of Formula (I) prepared according to a process described in the present application has an endothermic peak at about 120° C. in a DSC thermogram. For example, ranolazine of Formula (I) prepared according to a process described in the present application has a DSC thermogram substantially as illustrated in FIG. 2.

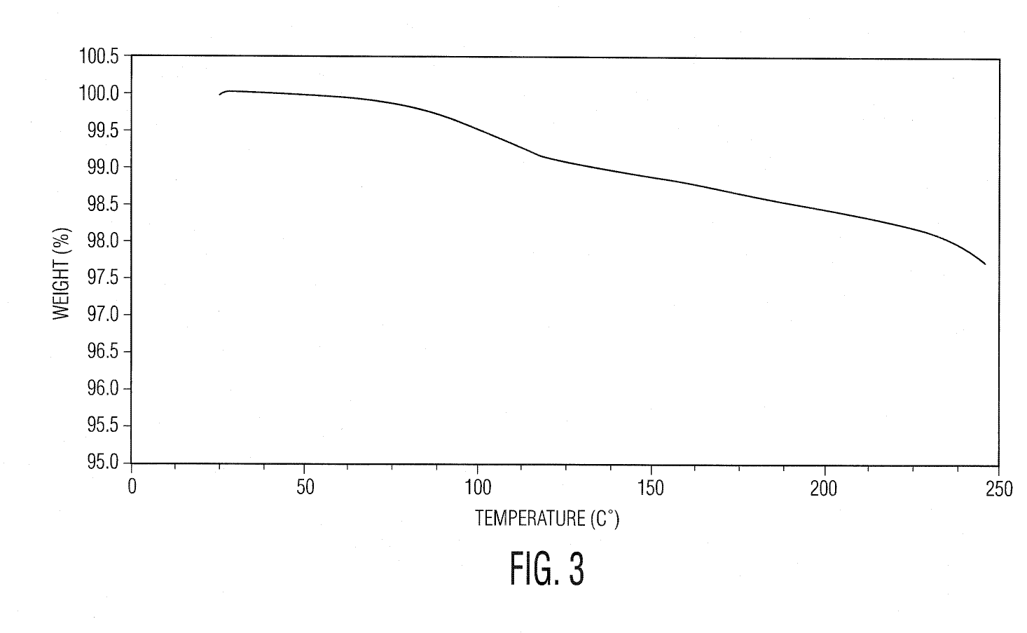

For example, ranolazine of Formula (I) prepared according to a process described in the present application has a TGA curve corresponding to a weight loss of less than about 3% w/w. For example, ranolazine of Formula (I) prepared according to a process described in the present application has a TGA curve substantially as illustrated in FIG. 3.

The present application also includes physical characteristics, such as, for example, particle size distributions, bulk densities, and water content, of ranolazine of Formula I.

In an embodiment, the present application provides ranolazine having particle sizes less than about 150 μm, or less than about 100 μm, or less than about 50 μm, or less than about 20 μm, or less than about 10 μm.

For example, the present application provides ranolazine having a particle size distribution wherein the 10^{th} volume percentile particle size (D₁₀) is less than about 15 μm, the 50^{th} volume percentile particle size (D₅₀) is less than about 35 μm, and/or the 90^{th} volume percentile particle size (D₉₀) is less than about 60 μm.

For example, the present application provides ranolazine having a particle size distribution wherein the 10^{th} volume percentile particle size (D₁₀) is less than about 5 μm, the 50^{th} volume percentile particle size (D₅₀) is less than about 10 μm, and/or the 90^{th} volume percentile particle size (D₉₀) is less than about 20 μm. The “10^{th} volume percentile” as used herein, unless otherwise defined refers to the size of particles, below which 10% of the measured particle volume lies; “50^{th} volume percentile” as used herein, unless otherwise defined refers to the size of particles, below which 50% of the measured particle volume lies, and “90^{th} volume percentile” as used herein, unless otherwise defined refers to the size of particles, below which 90% of the measured particle volume lies.

In an embodiment, the present application provides ranolazine having a particle size distribution span of less than about 3 or less than about 2.

Particle size distributions of ranolazine particles may be measured by any technique known in the art. For example, particle size distributions of ranolazine particles may be measured using light scattering equipment, such as, for example, a Malvern Master Sizer 2000 from Malvern Instruments Limited, Malvern, Worcestershire, United Kingdom (helium neon laser source, ranolazine suspended in light liquid paraffin, size range: 0.01 μm to 3000 μm).

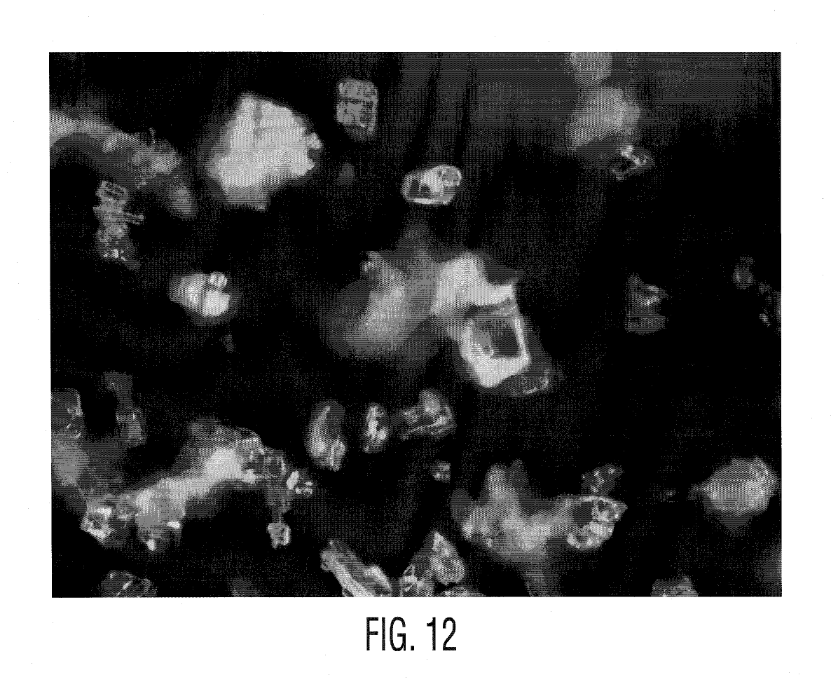

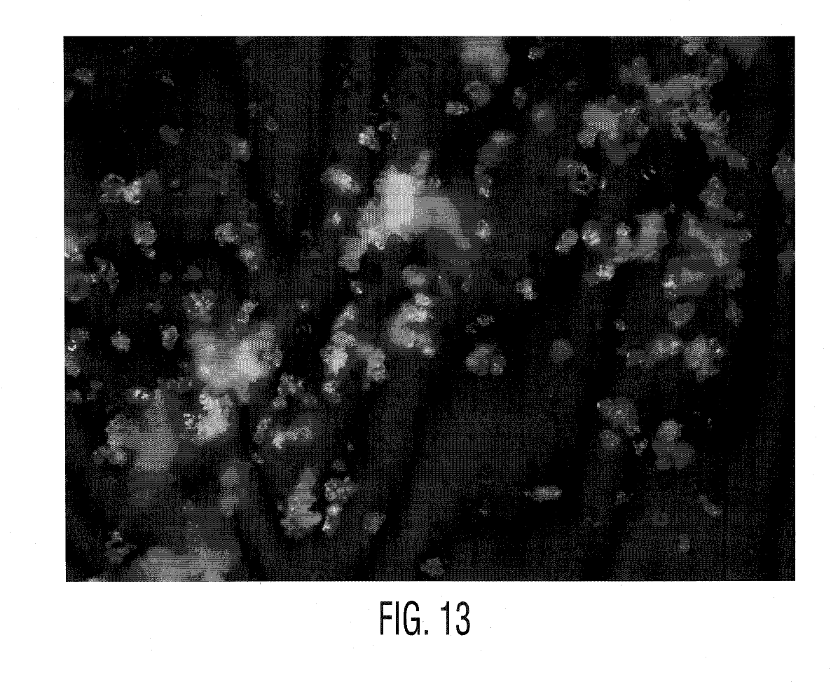

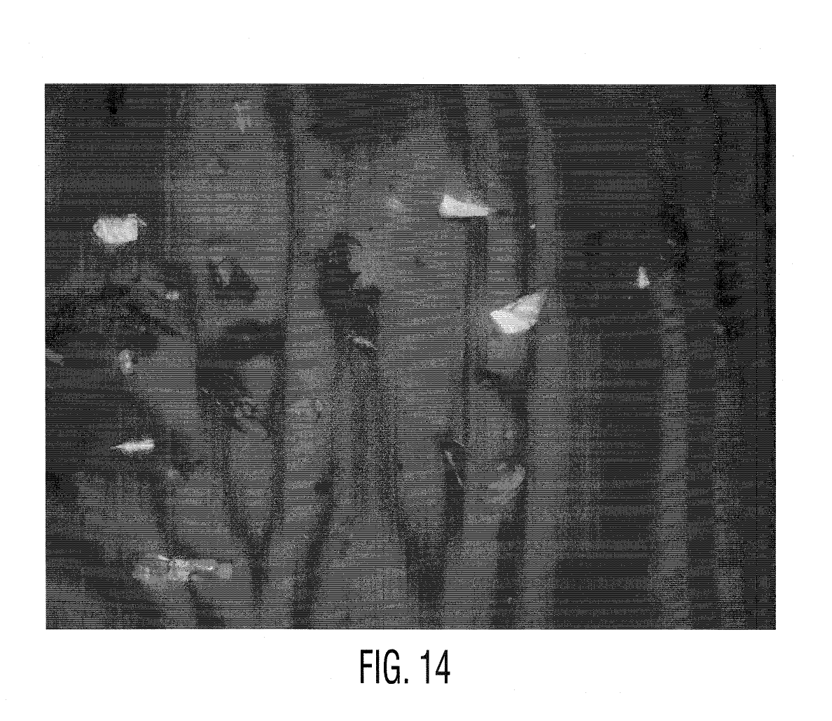

In an embodiment, the present application provides ranolazine having a particle shape substantially as pictured in any of FIGS. 12, 13, and 14.

In an embodiment, the present application provides a pharmaceutical composition comprising ranolazine having a specific surface area more than about 0.1 m²/g, or more than about 0.5 m²/g, or more than about 1 m²/g, or more than about 2 m²/g, or more than about 3 m²/g, or more than about 5 m²/g. “Specific surface area” as used herein, unless otherwise defined refers to the total particle surface of 1 gram of particles of a given material per square meter of particle surface area. The specific surface area of ranolazine of the present invention may be measured by a BET (Brunauer, Emmett and Teller) specific surface method, such as using a Micromeritics Gemini surface area analyzer, model 2365. Samples for analysis are degassed at 40° C. under reduced pressure and the determination of the adsorption of nitrogen gas at 77° K may be measured for relative pressures in the range of 0.05-0.3. Specific surface area and span of ranolazine of the present application may be measured by using light scattering equipment, such as, for example, a Malvern Master Sizer 2000 (helium neon laser source, ranolazine suspended in light liquid paraffin, size range: 0.01 μm to 3000 μm).

In an embodiment, the present application provides ranolazine having bulk densities less than about 0.8 g/mL, or less than about 0.5 g/mL, or less than about 0.3 g/mL. Bulk density may be determined using Test 616 “Bulk Density and Tapped Density,” as in United States Pharmacopoeia 29, United States Pharmacopeial Convention, Inc., Rockville, Md., 2005, in method 2.

In an embodiment, the present application also provides ranolazine having a water content of less than about 5%, or less than about 3%, or less than about 2%, or less than about 1%, or less than about 0.5%, by weight as measured by the Karl Fischer method. Water content is expressed in % by weight, which refers to percentage weight of water with respect to the total weight of the sample when analyzed by the Karl Fischer method.

In an embodiment, the present application also provides process for packaging and storing of ranolazine with increased stability and shelf life, which processes comprise storing ranolazine within a sealed clear polythene bag flushed with nitrogen, which first bag is sealed, along with a silica gel desiccant pouch, within a black polythene bag filled with nitrogen, which second bag is sealed, along with a silica gel pouch, within a triple laminated bag, which third bag is sealed within an HDPE container held in controlled environment chamber.

In an embodiment, the present application also provides pharmaceutical compositions prepared using ranolazine having particle sizes less than about 150 μm, or less than about 100 μm, or less than about 50 μm, or less than about 20 μm, or less than about 10 μm, together with one or more pharmaceutically acceptable excipients.

In an embodiment, the present application also provides pharmaceutical compositions comprising ranolazine or a pharmaceutically acceptable salt thereof prepared by processes of the present invention, together with one or more pharmaceutically acceptable excipients.

For example, the present application includes pharmaceutical compositions prepared using ranolazine that, prior to formulation, had a bulk density of less than about 0.8 g/mL, or less than about 0.5 g/mL, or less than about 0.3 g/mL, together with one or more pharmaceutically acceptable excipients.

For example, the present application includes pharmaceutical composition prepared using ranolazine that, prior to formulation, had a specific surface area greater than about 0.1 m²/g, or greater than about 0.5 m²/g, or greater than about 1 m²/g, or greater than about 2 m²/g, or greater than about 3 m²/g, or greater than about 5.0 m²/g, together with one or more pharmaceutically acceptable excipients.

A pharmaceutical composition comprising ranolazine or a pharmaceutically acceptable salt thereof with one or more pharmaceutically acceptable excipients may be formulated as solid oral dosage forms, such as, for example, powders, granules, pellets, tablets, and capsules; liquid oral dosage forms, such as, for example, syrups, suspensions, dispersions, and emulsions; and injectable preparations, such as, for example, solutions, dispersions, and freeze dried compositions. Immediate release compositions may be conventional, dispersible, chewable, mouth dissolving, or flash melt preparations. Modified release compositions may comprise hydrophilic and/or hydrophobic release rate controlling substances to form matrix and/or reservoir systems. The pharmaceutical compositions may be prepared by direct blending, dry granulation, or wet granulation or by extrusion and spheronization. Compositions may be presented as uncoated, film coated, sugar coated, powder coated, enteric coated, or modified release coated.

Pharmaceutical compositions according to the present application comprise one or more pharmaceutically acceptable excipients. Pharmaceutically acceptable excipients include and are not limited to: diluents, such as, for example, starch, pregelatinized starch, lactose, powdered cellulose, microcrystalline cellulose, dicalcium phosphate, tricalcium phosphate, mannitol, sorbitol, sugar, and the like; binders, such as, for example, acacia, guar gum, tragacanth, gelatin, polyvinylpyrrolidones, hydroxypropyl celluloses, hydroxypropyl methylcelluloses, pregelatinized starches, and the like; disintegrants, such as, for example, starch, sodium starch glycolate, pregelatinized starch, crospovidones, croscarmellose sodium, colloidal silicon dioxide, and the like; lubricants, such as, for example, stearic acid, magnesium stearate, zinc stearate, and the like; glidants, such as, for example, colloidal silicon dioxide and the like; solubility or wetting enhancers, such as, for example, anionic or cationic or neutral surfactants; complex forming agents, such as, for example, various grades of cyclodextrins; release rate controlling agents, such as, for example, hydroxypropyl celluloses, hydroxymethyl celluloses, hydroxypropyl methylcelluloses, ethyl celluloses, methyl celluloses, various grades of methyl methacrylates, waxes, and the like. Other pharmaceutically acceptable excipients that are of use include, but are not limited to, film formers, plasticizers, colorants, flavoring agents, sweeteners, viscosity enhancers, preservatives, antioxidants, and the like.

The amount of ranolazine in each unit dosage form on a free weight basis can range from about 50 to about 2000 mg, or from about 100 to about 1500 mg, or from about 500 to about 1000 mg. Dosage forms can be administered in a single daily dose or divided throughout the day. Typical doses include 500 mg twice a day and 1000 mg twice a day. Controlled release dosage forms may be employed to provide twice a day or even once a day dosing.

Certain specific aspects and embodiments of the present application will be explained in more detail with reference to the following examples, which are provided for purposes of illustration only and should not be construed as limiting the scope of the present application in any manner.

### Comparative Example A

### Preparation of 1-(2-methoxyphenoxy)-2,3-epoxypropane According to U.S. Pat. No. 4,567,264

1,4-dioxane (65 mL) and water (19.5 mL) are charged into a round-bottom flask and stirred for 5-10 minutes at 25-35° C. Sodium hydroxide (9.7 g) is added and the mixture is stirred for 15-20 minutes. 2-methoxyphenol (25 g) is added at 25-35° C. and stirred for 10-15 minutes. Epichlorohydrin (26 g) is added, the temperature is raised to 90° C., and the mixture is maintained for 1-2 hours, then cooled to 25-35° C. Diethyl ether (50 mL) is added and the layers are separated. The organic layer is washed with water (2×50 mL) and the solvent from the organic layer is distilled under reduced pressure at 50-55° C., to afford 33.8 g of the title compound.

Purity by HPLC: 50.48%; dimer impurity of Formula IIa: 43.37%; chloro impurity of Formula IIb: 0.79%; dihydroxy impurity of Formula IIc: 2.74%.

### Comparative Example B

### Preparation of 1-(2-methoxyphenoxy)-2,3-epoxypropane According to International Application Publication No. WO 2008/047388 A2

2-methoxyphenol (100 g) and toluene (800 mL) are charged into a round-bottom flask and stirred for 5-10 minutes. Tetrabutylammonium bromide (20 g) and a solution of sodium hydroxide (40 g) in water (200 mL) are added and the mixture is stirred at 25-35° C. for 30 minutes, then epichlorohydrin (100 g) is slowly added. The mixture is maintained at 35-40° C. for 6 hours and cooled to 25-35° C. The layers are separated and the aqueous layer is extracted with toluene (200 mL). The combined organic layer is washed with sodium chloride solution, mixed with charcoal, and filtered. The solvent is distilled from the organic layer at 65-70° under reduced pressure, to afford 136.4 g of the title compound.

Purity by HPLC: 58.36%; dimer impurity of Formula IIa: 23.18%; chloro impurity of Formula IIb: 1.31%; dihydroxy impurity of Formula IIc: 5.3%.

### Comparative Example C

### Preparation of 1-(2-methoxyphenoxy)-2,3-epoxypropane

2-methoxyphenol (25 g) and water (100 mL) are charged into a round-bottom flask and stirred for 5-10 minutes. A solution of sodium hydroxide (4.0 g) and sodium bicarbonate (8.5 g) in water (25 mL) is added at 25-35° C. The mixture is stirred for 30-45 minutes at 25-35° C. and epichlorohydrin (55.9 g) is slowly added at 25-35° C. The mixture is maintained at 25-35° C. for 13-14 hours and the layers are separated. The organic layer, containing the product and unreacted epichlorohydrin, is distilled below 90° C., to afford 35.9 g of the title compound.

Purity by HPLC: 76.85%; dimer impurity of Formula IIa: 0.086%; chloro impurity of Formula IIb: 20.04%; dihydroxy impurity of Formula IIc: 1.635%.

### Example 1

### Preparation of 1-(2-methoxyphenoxy)-2,3-epoxypropane

2-methoxyphenol (100 g) and water (400 mL) are charged into a round-bottom flask and stirred for 5-10 minutes. A solution of sodium hydroxide (16.1 g) in water (100 mL) is added at 25-35° C. and stirred for 45-60 minutes. Epichlorohydrin (223.5 g) is added at 25-35° C. and the mixture is maintained for 10-12 hours. Layers are separated. Water (400 mL) and a solution of sodium hydroxide (32.2 g) in water (100 mL) are added to the organic layer containing the product. The mixture is maintained at 25-35° C. for 5-6 hours. The layers are separated and 10% sodium hydroxide solution (300 mL) is added to the organic layer containing the product at 25-35° C. The mass is stirred for 20-30 minutes and layers are separated. The organic layer containing the product is distilled at 85-89° C. under reduced pressure, to afford 136.5 g of the title compound.

Purity by HPLC: 98.28%; dimer impurity of Formula IIa: 0.29%; chloro impurity of Formula IIb: 0.15%; dihydroxy impurity of Formula IIc: 0.70%.

### Example 2

### Preparation of 1-[3-(2-methoxyphenoxy)-2-hydroxypropyl]piperazine

Methanol (250 mL) and piperazine (96 g) are charged into a round-bottom flask and stirred for 5-10 minutes to dissolve piperazine completely. The solution is cooled to 0-5° C. 1-(2-methoxyphenoxy)-2,3-epoxypropane (50 g) is slowly added and the mixture is maintained at 0-5° C. for 2-3 hours. The mixture is charged into water (200 mL) and stirred at 25-35° C. for 10-15 minutes. The mass is filtered and the filtrate is extracted with dichloromethane (5×50 mL). Acetic acid (32.5 mL) and water (200 mL) are added to the organic layer and stirred for 5-10 minutes, then the layers are separated. The aqueous layer is made basic with aqueous ammonia (55 mL) and then is extracted with dichloromethane (5×50 mL). The solvent from the organic layer is distilled completely under reduced pressure at 40-45° C., to afford 44.2 g of the title compound.

Purity by HPLC: 95.714%.

### Example 3

### Preparation of 2-chloro-N-(2,6-dimethylphenyl) acetamide

2,6-dimethylaniline (100 g) and dichloromethane (500 mL) are charged into a round-bottom flask and stirred for 5-10 minutes. Sodium carbonate (43.8 g) is added and the mixture is cooled to 10-15° C. Chloroacetyl chloride (79 mL) is slowly added at 10-15° C. and the mixture is maintained at 10-15° C. for 60-90 minutes. The temperature is raised to 25-35° C. and water (1000 mL) is added. The organic solvent is evaporated completely at 40-45° C. under reduced pressure. The residue is cooled to 25-35° C. and maintained for 45-60 minutes. The obtained solid is filtered and washed with water (200 mL), then the solid is dried at 70° C., to afford 150 g of the title compound.

Purity by HPLC: 98.95%.

### Example 4

### Purification of 2-chloro-N-(2,6-dimethylphenyl) acetamide

2-chloro-N-(2,6-dimethylphenyl) acetamide (17 g) and toluene (50 mL) are charged into a round-bottom flask and stirred for 5-10 minutes. The mixture is heated to 80-85° C. to dissolve 2-chloro-N-(2,6-dimethylphenyl) acetamide completely. The solution is maintained at 80-85° C. for 30-45 minutes and then cooled to 25-35° C. The solution is further cooled to 0-5° C. and maintained for 30-45 minutes. The formed solid is filtered, washed with toluene (15 mL), and dried at 75° C., to afford 15.3 g of the title compound.

Purity by HPLC: 99.60%.

### Example 5

### Preparation of N-(2,6-dimethylphenyl)-1-piperazine acetamide

2-chloro-N-(2,6-dimethylphenyl) acetamide (100 g), piperazine (182 g) and methanol (300 mL) are charged into a round-bottom flask and stirred for 5-10 minutes. The mixture is heated to reflux temperature and maintained for 2-3 hours. The mixture is cooled to 25-35° C. and water (800 mL) is added. The mixture is stirred for 15-30 minutes, then filtered to remove unwanted solid, and the filter is washed with water (200 mL). Dichloromethane (400 mL) is added to the filtrate and the mixture is stirred for 15-30 minutes. The layers are separated and the aqueous layer is extracted with dichloromethane (400 mL). The combined organic layer is washed with a solution of sodium hydroxide (20 g) in water (350 mL) and the solvent is evaporated at 40-45° C. 500 mL of n-hexane is added to the residue at 25-35° C. and the mixture is maintained for 30-45 minutes. The solid is filtered under reduced pressure, washed with n-hexane (100 mL), and dried at 40° C., to afford 88.5 of the title compound.

Purity by HPLC: 99.61%.

### Example 6

### Preparation of Ranolazine

Preparation A. N-(2,6-dimethylphenyl)-1-piperazine acetamide (20 g), 1-(2-methoxyphenoxy)-2,3-epoxypropane (19.2 g), and acetone (280 mL) are charged into a round-bottom flask and stirred for 5-10 minutes. The mixture is heated to reflux temperature and maintained for 15-16 hours. The mixture is cooled to 25-30° C., further cooled to 20±2° C., maintained at 20±2° C. for 1 hour, and the formed solid is filtered and washed with acetone (2×20 mL). The solid is dried at 60-65° C., to afford the 22.3 g of the title compound. Purity by HPLC: 97.98%.

Preparation B. N-(2,6-dimethylphenyl)-1-piperazine acetamide (20 g), 1-(2-methoxyphenoxy)-2,3-epoxypropane (19 g), and acetonitrile (200 mL) are charged into a round-bottom flask and stirred for 5-10 minutes. The mixture is heated to reflux temperature and maintained for 9-10 hours. The mixture is cooled to 0-5° C., maintained for 1 hour, and the formed solid is filtered and washed with acetonitrile (20 mL). The solid is dried at 60-65° C., to afford 29.7 g of the title compound. Purity by HPLC: 98.56%.

Preparation C. N-(2,6-dimethylphenyl)-1-piperazine acetamide (20 g), 1-(2-methoxyphenoxy)-2,3-epoxypropane (19 g), and ethyl acetate (200 mL) are charged into a round-bottom flask and stirred for 5-10 minutes. The mixture is heated to reflux temperature and maintained for 13-14 hours. The reaction mixture is cooled to 0-5° C. and maintained for 45-60 minutes, then the formed solid is filtered and washed with ethyl acetate (20 mL). The solid is dried at 60-65° C., to afford 30.6 g of the title compound. Purity by HPLC: 94.21%.

Preparation D. N-(2,6-dimethylphenyl)-1-piperazine acetamide (20 g), 1-(2-methoxyphenoxy)-2,3-epoxypropane (19 g), ethyl acetate (100 mL), and water (100 mL) are charged into a round-bottom flask and stirred for 5-10 minutes. The mixture is heated to reflux temperature and maintained for 10-11 hours. The mixture is cooled to 0-5° C. and maintained for 45-60 minutes. The formed solid is filtered and washed with ethyl acetate (20 mL). The solid is dried at 60-65° C., to afford 27.6 g of the title compound. Purity by HPLC: 97.86%.

Preparation E. N-(2,6-dimethylphenyl)-1-piperazine acetamide (10 g), 1-(2-methoxyphenoxy)-2,3-epoxypropane (9.5 g), and water (100 mL) are charged into a round-bottom flask and stirred for 5-10 minutes. The mixture is heated to reflux temperature and maintained for 9-10 hours. The mixture is cooled to 0-5° C. and maintained for 45-60 minutes. Acetone (50 mL) is added and the mixture is maintained at 0-5° C. for 10-12 hours. The formed solid is filtered and washed with acetone (10 mL). The solid is dried at 60-65° C., to afford 8.4 g of the title compound. Purity by HPLC: 72.77%.

Preparation F. N-(2,6-dimethylphenyl)-1-piperazine acetamide (20 g), 1-(2-methoxyphenoxy)-2,3-epoxypropane (19 g), and isopropanol (200 mL) are charged into a round-bottom flask and stirred for 5-10 minutes. The mixture is heated to reflux temperature and maintained for 9-10 hours. The mixture is cooled to 0-5° C. and maintained for 45-60 minutes. Acetone (50 mL) is added and the mixture is maintained at 0-5° C. for 45-60 minutes. The formed solid is filtered and washed with chilled isopropanol (20 mL). The solid is dried at 60-65° C. under reduced pressure, to afford 24.4 g of the title compound. Purity by HPLC: 93.947%.

Preparation G. N-(2,6-dimethylphenyl)-1-piperazine acetamide (20 g), 1-(2-methoxyphenoxy)-2,3-epoxypropane (19 g), and methanol (200 mL) are charged into a round-bottom flask and stirred for 5-10 minutes. The mixture is heated to reflux temperature and maintained for 6 hours. The solvent is evaporated at 60-65° under reduced pressure and the residue is cooled to 25-35° C. Acetone (100 mL) is added to the residue and the mixture is cooled to 0-5° C. and maintained for 45-60 minutes, then the formed solid is filtered and washed with chilled acetone (20 mL). The solid is dried at 60-65° C. under reduced pressure to afford 29.5 g of the title compound. Purity by HPLC: 95.96%.

### Example 7

### Preparation of Ranolazine Dihydrochloride

2-chloro-N-(2,6-dimethylphenyl) acetamide (25.8 g), 1-[3-(2-methoxyphenoxy)-2-hydroxypropyl]piperazine (30 g), and dimethylformamide (200 mL) are charged into a round-bottom flask and stirred for 5-10 minutes. The mixture is heated to 90-95° C. and maintained for 4-5 hours. The mixture is cooled to 25-35° C. and is added to water (300 mL). The mixture is acidified with HCl (10 mL), washed with toluene (2×100 mL), and the aqueous layer is made basic with aqueous ammonia (40 mL). The aqueous layer is extracted with dichloromethane (3×180 mL) and the solvent from the combined organic layer is distilled completely under reduced pressure at 40-55° C. The residue is cooled to 25-35° C. Isopropanol, HCl [10-13%] (100 mL), and acetone (300 mL) are added to the residue and stirred for 5-10 minutes. The mixture is heated to 60-65° C. and maintained for 15-30 minutes. The mixture is cooled to 0-5° C. and maintained for 45-60 minutes. The formed solid is filtered and washed with a mixture of isopropanol, HCl, and acetone (15 mL; 1:1). The solid is dried at 40-45° C., to afford 33.7 g of the title compound.

### Example 8

### Preparation of Ranolazine from Ranolazine Dihydrochloride

Ranolazine dihydrochloride (5 g) and water (25 mL) are charged into a round-bottom flask and stirred for 5-10 minutes. A 40% sodium hydroxide solution (10 mL) is added and the mixture is stirred for 5-10 minutes. Dichloromethane (25 mL) is added at 25-35° C. and stirred for 10-15 minutes. The layers are separated and the aqueous layer is extracted with dichloromethane (2×5 mL). The combined organic layer is washed with water (10 mL) and the solvent from the organic layer is evaporated completely under reduced pressure at 35-40° C. The residue is cooled to 25-35° C. Acetone (5 mL) is added to the residue and the solvent is evaporated completely at 40-45° C. The residue is cooled to 25-35° C. and acetone (15 mL) is added. The mixture is cooled to 0-5° C. and maintained for 30-45 minutes. The solid is filtered, washed with acetone (5 mL), and dried at 60-65° C., to afford the 3.3 g of the title compound. Purity by HPLC: 99.36%.

### Example 9

### Purification of Ranolazine

Ranolazine (25 g) and acetone (250 mL) are charged into a round-bottom flask and stirred for 5-10 minutes. The mixture is heated to reflux temperature and maintained for 15-30 minutes. The solution is cooled to 25-35° C. and maintained for 45-60 minutes. The formed solid is filtered, washed with acetone (25 mL), and suction dried for 15-30 minutes. Acetone (150 mL) is charged to the wet solid and stirred for 5-10 minutes. The mixture is heated to reflux temperature and maintained for 15-30 minutes. The solution is cooled to 25-35° C. and maintained for 45-60 minutes. The formed solid is filtered, washed with acetone (15 mL), and dried at 60-65° C., to afford the 18.2 g of the title compound. Purity by HPLC: 99.89%.

### Example 10

### Purification of Ranolazine

Ranolazine (10 g) and methanol (10 mL) are charged into a round-bottom flask and stirred for 5-10 minutes. The mixture is heated to reflux temperature and maintained for 10-15 minutes. The solution is cooled to 25-35° C. and maintained for 30-45 minutes. Acetone (50 mL) is added and the mixture is maintained at 25-35° C. for 45-60 minutes. The formed solid is filtered, washed with acetone (20 mL), and dried at 60-65° C., to afford 7.0 g of the title compound. Purity by HPLC: 99.89%.

### Example 11

### Preparation of 2-chloro-N-(2,6-dimethylphenyl) acetamide

2,6-dimethylaniline (25 g) and ethyl acetate (75 mL) are charged into a round-bottom flask and stirred for 5-10 minutes. Sodium carbonate (26 g) is added and the mixture is cooled to 0-5° C. Chloroacetyl chloride (24.6 mL) is slowly added at 0-5° C. and the mixture is maintained at 0-5° C. for 2-3 hours. Water (100 mL) is added and the mixture is maintained at 0-5° C. for 30-45 minutes. The formed solid is filtered and washed with water (50 mL). The solid is dried at 70-80° C., to afford 35 g of the title compound.

Purity by HPLC: 98.12%; dichloroacetyl impurity of Formula IIIa: 0.43%.

### Example 12

### Preparation of 2-chloro-N-(2,6-dimethylphenyl) acetamide

2,6-dimethylaniline (25 g) and toluene (75 mL) are charged into a round-bottom flask and stirred for 5-10 minutes. Sodium carbonate (26 g) is added and the mixture is cooled to 0-5° C. Chloroacetyl chloride (24.6 mL) is slowly added at 0-5° C. and the mixture is maintained at 0-5° C. for 3-4 hours. Water (100 mL) is added and the mixture is maintained at 0-5° C. for 30-45 minutes. The formed solid is filtered and washed with water (50 mL). The solid is dried at 70-80° C., to afford 33.5 g of the title compound.

Purity by HPLC: 97.23%; dichloroacetyl impurity of Formula (IIIa): 0.74%.

### Example 13

### Preparation of N-(2,6-dimethylphenyl)-1-piperazine acetamide

2-chloro-N-(2,6-dimethylphenyl) acetamide (25 g), piperazine (32.9 g) and methanol (75 mL) are charged into a round-bottom flask and stirred for a period of 5-10 minutes. The reaction mixture is heated to a temperature of 60-65° C. and maintained for a period of 1-2 hours. The reaction mixture is distilled at a temperature of 60-65° C. up to 80% of the reaction mixture and cooled to 25-35° C. The reaction mixture is maintained for a period of 30-45 minutes and filtered under reduced pressure on diatomaceous earth followed by washing the diatomaceous earth with water (75 mL). The reaction mass is extracted from the obtained filtrate into dichloromethane (200 mL). The obtained organic layer is washed with water (75%) and the solvent from the organic layer is evaporated at a temperature of 40-45° C. to 80% of the reaction mass. 125 mL of n-hexane is charged to the resultant reaction mass and distilled at a temperature of 60-65° C. to 30-40% of the reaction mass. The reaction mass is cooled to a temperature of 25-35° C. and maintained for a period of 45-60 minutes. The suspension is filtered under vacuum and washed the solid with n-hexane (25 mL). The resultant solid is dried at a temperature of 40-45° C. to afford 22.5 g of the title compound. {Purity by HPLC: 99.70%}

### Example 14

### Preparation of Ranolazine

Acetone (25 L), 1-[3-(2-methoxyphenoxy)-2-hydroxypropyl]-piperazine (4.75 kg) and N-(2,6-dimethylphenyl)-1-piperazine acetamide (5.0 kg) are charged into a reactor. The mass is heated to 56±2° C. and maintained at 56±2° C. for 18 hours. The mass is cooled to 2.5±2.5° C. and maintained for 3-4 hours. The mass is filtered and the solid is washed with chilled acetone (5 L). The solid is spin dried for 2 hours. The wet solid, acetone (28 L), and methanol (7 L) are charged into a round-bottom flask. The mass is heated to 54±2° C. and maintained for 45 minutes. The mass is cooled to 25±5° C., further cooled to 2.5±2.5° C., and maintained for 4 hours. The mass is filtered and the solid is washed with chilled acetone (5 L). The solid is spin dried for 1-2 hours and then dried at 70-75° C. under reduced pressure, to afford 5.88 kg of the title compound.

Particle size distribution: D₁₀: 6.311 μm; D₅₀: 22.490 μm; D_{go}: 47.539 μm. Specific surface area: 0.539 m²/g; span: 1.833.

### Example 15

### Preparation of Ranolazine

Example 14 is repeated, and 5.25 kg of the title compound are obtained.

Particle size distribution: D₁₀: 5.272 μm; D₅₀: 18.656 μm; D₉₀: 57.748 μm.

Specific surface area: 0.655 m²/g; span: 2.813.

### Example 16

### Purification of Ranolazine

Acetone (16.5 L), methanol (5.5 L), and ranolazine (5.5 kg) are charged into a reactor. The mass is heated to 55±2° C. and maintained for 40 minutes. The mass is cooled to 40-50° C., then is filtered and the solid is washed with acetone (5.5 L). The filtrate is cooled to 2.5±2.5° C. and maintained for 4 hours. The mass is filtered and the solid is washed with chilled acetone (5.5 L) and dried at 60-65° C. under reduced pressure, to afford 4.5 kg of the title compound.

Particle size distribution: D₁₀: 5.026 μm; D₅₀: 18.770 μm; D₉₀: 39.137 μm.

Specific surface area by Malvern method: 1.63 m²/g; span: 1.817 by Malvern method; specific surface area by BET method: 0.46 m²/g.

### Example 17

### Particle Size Reduction of Ranolazine

Ranolazine (2.0 kg), having a particle size distribution where d(0.1)=5.026 μm, d(0.5)=18.770 μm, and d(0.9)=39.137 μm, is micronized using a jet mill (MIDAS MIKRONIZER M-200 system GMP Model) with 4.0 kg/cm² of nitrogen pressure and at 2.5 kg/cm² of feed pressure. The micronized material is sifted through a 40 mesh sieve.

Particle size distribution: D₁₀: 1.324 μm; D₅₀: 5.140 μm; D₉₀: 11.116 μm.

Specific surface area by Malvern method: 1.99 m²/g; span by Malvern method: 1.905; specific surface area by BET method: 2.54 m²/g.

Purity by HPLC: compound of Formula (II): 2.39 ppm; compound of Formula (III):1.86 ppm; chloro impurity of Formula IIb: 0.74 ppm; dichloroacetyl impurity of Formula IIIa: not detected.

Purity by GC: 2,6-dimethylaniline of Formula (VII): not detected; epichlorohydrin of Formula (VIII): not detected.

### Example 18

### Purification of Ranolazine

Ranolazine (250.0 g), acetone (1 L), and methanol (250 mL) are charged into a round-bottom flask and stirred for 5 minutes. The mixture is heated to 56° C. and stirred for 30 minutes. The solution is filtered and the filtrate is charged into a round-bottom flask. The solution is cooled to 2-4° C. and maintained for 45 minutes. The solid is filtered, washed with chilled acetone (250 mL), and dried at 74° C., to afford 210 g of the title compound.

Purity by HPLC: 99.946%; water content: 0.2%; ROI (residue on ignition): 0.053%; bulk density before tapping: 0.353 g/mL; bulk density after tapping: 0.477 g/mL; methanol content: 200 ppm; acetone content: 476 ppm; toluene content: 14.5 ppm by a gas chromatography method.

Particle size distribution: D₁₀: 4.420 μm; D₅₀: 16.147 μm; D₉₀: 30.439 μm.

Specific surface area: 0.705 m²/g; span: 1.611.

### Example 19

### Purification of Ranolazine

Ranolazine (100.0 g), methanol (100 mL), and acetone (400 mL) are charged into an automated reactor at 27° C. The mixture is heated to 55° C. at 1.8° C. per minute. The mixture is cooled to 0° C. at −1.5° C. per minute between 55° C. and 30° C., and −0.2° C. per minute between 30° C. and 0° C. The mixture is maintained at 0° C. for 2-3 hours. The solid is filtered, washed with chilled acetone (100 mL), and dried at 70° C., to afford 80.0 g of the title compound.

Particle size distribution: D₁₀: 13.262 μm; D₅₀: 41.574 μm; D₉₀: 89.060 μm.

Specific surface area: 0.291 m²/g; span: 1.823.

### Example 20

### Preparation of N-(2,6-dimethylphenyl)-1-piperazine acetamide

Preparation A. 2-Chloro-N-(2,6-dimethylphenyl) acetamide (75.0 g), piperazine (98.0 g), and methanol (225 mL) are charged into a round-bottom flask at 26° C. and stirred for 5 minutes. The mixture is heated to 65° C. and maintained for 3-4 hours. The mixture is distilled at 65° C. and the residue is cooled to 25° C. Water (600 mL) is added at 25° C. and stirred for of 55 minutes. The unwanted solid is filtered through diatomaceous earth and washed with water (225 mL). The filtrate volume is 825 mL.

A 275 mL portion of the filtrate is charged into a round-bottom flask and the pH is adjusted to 5.8 with 44% phosphoric acid solution (38 mL), then the mixture is stirred for 15 minutes. The unwanted solid is filtered and the filtrate is washed with water (25 mL). The pH is adjusted to 10.4 with a solution of sodium hydroxide (20%; 40 mL). The aqueous layer is extracted with dichloromethane (2×125 mL). The total organic layer is washed with water (50 mL) and the solvent is evaporated at 40° C. to 80% of the initial volume. 125 mL of n-hexane is added at 30° C. and the solvent is evaporated at 55° C. to 40% of the starting volume. The mass is stirred at 25° C. for 45 minutes. The formed solid is filtered, washed with n-hexane (25 mL), and dried at 55° C., to afford 21.1 g of the title compound.

Purity by HPLC: 99.725%; piperazine content: 0.008%.

Preparation B. 2-Chloro-N-(2,6-dimethylphenyl) acetamide (100.0 g), piperazine (130.6 g), and methanol (300 mL) are charged into a round-bottom flask at 26° C. and stirred for 5 minutes. The mixture is heated to 65-68° C. and maintained for 6-7 hours. The mixture is distilled at 65° C. and the residue is cooled to 25° C. Water (800 mL) is added to the residue at 25° C. and stirred for 40 minutes. The unwanted solid is filtered and washed with water (300 mL). The filtrate volume is 1100 mL.

A 275 mL portion of the filtrate is charged into a round-bottom flask and the pH is adjusted to 5.4 with 44% phosphoric acid solution (35 mL) at 25° C. and stirred at 25° C. for 30 minutes. The unwanted solid is filtered and the filtrate is washed with water (25 mL). The filtrate pH is adjusted to 10.6 with a solution of sodium hydroxide (20%; 40 mL). Dichloromethane (50 mL) is added and stirred for 5 minutes. The layers are separated. The aqueous layer is extracted with dichloromethane (2×125 mL) and the total organic layer is washed with water (75 mL). The solvent from the organic layer is evaporated at 40° C. to 80% of the initial volume. Cyclohexane (125 mL) is added at 40° C. and the solvent is evaporated at 64° C. to 20% of the starting volume. The mass is stirred at 25° C. for 1 hour, 10 minutes. The obtained solid is filtered, washed with cyclohexane (25 mL), and then dried at 55° C., to afford 20.8 g of the title compound.

Purity by HPLC: 99.537%.

### Example 21

### Preparation of N-(2,6-dimethylphenyl)-1-piperazine acetamide

Preparation A. Methanol (16 L) and piperazine (5.26 kg) are charged into a reactor and stirred for 5-10 minutes. 2-Chloro-N-(2,6-dimethylphenyl) acetamide (4.0 kg) is added and stirred for 10-15 minutes. The mixture is heated to 60-65° C. and maintained at 67.5±2.5° C. for 3-4 hours. The mixture is cooled to 10-15° C. and maintained for 35 minutes. The mixture is filtered under a nitrogen atmosphere and the unwanted solid is washed with methanol (4 L) at 10-15° C. The filtrate is charged into a reactor and the solvent is evaporated completely below 70° C. under reduced pressure. Water (32 L) is added. The mass is cooled to 10-15° C. and maintained for 45 minutes. The mass is filtered through diatomaceous earth and washed with water (10.5 L). Dichloromethane (16 L) is added to the filtrate and stirred for a period of 25 minutes. The layers are separated and the aqueous layer is extracted with dichloromethane (16 L). The combined organic layer is washed with water (12 L) and the solvent is evaporated completely below 50° C. under reduced pressure. The residue is cooled to 25-35° C. Hexane (12 L) is added to the residue and maintained for 90 minutes. The mass is filtered and the solid is washed with hexane (4 L). The solid is suction dried for 1 hour and then dried at 40-45° C. under reduced pressure, to afford 3.11 kg of the title compound in polymorphic crystalline form A.

Water content: 0.72% (w/w); ROI (residue on ignition): 0.07%.

Preparation B. Methanol (19.5 L) and piperazine (8.554 kg) are charged into a reactor and stirred for 5-10 minutes. 2-Chloro-N-(2,6-dimethylphenyl) acetamide (6.5 kg) is added and stirred for 10-15 minutes. The mixture is heated to 60-65° C. and maintained for 3 hours. The solvent is evaporated below 70° C. at atmospheric pressure. The residue is cooled to 25-35° C. Water (52 L) is added to the residue and the mixture is maintained at 25-35° C. for 35 minutes. The mass is filtered through diatomaceous earth and washed with water (13 L). The filtrate is charged into a reactor and dichloromethane (26 L) is added, then the mixture is stirred for 20 minutes and the layers are separated. The aqueous layer is extracted with dichloromethane (2×6.5 L) and the combined organic layer is washed with water (6.5 L). The solvent from the organic layer is evaporated to 80% of the initial volume below 50° C., is cooled to 25-35° C., and hexane (32.5 L) is added. The dichloromethane is evaporated completely below 60° C. The residue is cooled to 25-30° C. and maintained for 90 minutes. The formed solid is filtered, washed with hexane (6.5 L), and dried at 40-45° C. under reduced pressure, to afford 5.5 kg of the title compound, in polymorphic crystalline Form B.

Purity by HPLC: 99.29%; ROI (residue on ignition): 0.03% (w/w); water content: 1.06% (w/w).

### Example 22

### Preparation of Ranolazine

N-(2,6-dimethylphenyl)-1-piperazine acetamide (250 g) and acetone (1250 mL) are charged at 28° C. into a round-bottom flask and stirred for 5 minutes. 1-(2-methoxyphenoxy)-2,3-epoxypropane (236.8 g) is added. The mixture is heated to 55° C. and maintained for 15-16 hours. The mixture is cooled to 2-3° C. and maintained for 3-4 hours. The solid is filtered, washed with acetone (250 mL), and dried at 65° C., to afford 368.1 g of the title compound.

Purity by HPLC: 98.22%.

The compound (20.0 g), methanol (20 mL), and acetone (80 mL) are charged into a round-bottom flask and stirred for 10 minutes. The mixture is heated to 56° C. and stirred for 30 minutes. The solution is cooled to 4-5° C. and stirred for 3-4 hours. The formed solid is filtered under reduced pressure, washed with acetone (20 mL), and dried at 72° C., to afford 17.1 g of the title compound.

Purity by HPLC: 99.749%; dimer impurity of Formula (Va): 0.125%.

### Example 23

### Preparation of Ranolazine

N-(2,6-dimethylphenyl)-1-piperazine acetamide (20 g), 1-(2-methoxyphenoxy)-2,3-epoxypropane (18.9 g) and acetone (100 mL) are charged at 26° C. into a round-bottom flask and stirred for 5 minutes. The mixture is heated to 55° C. and maintained for 16-17 hours. The mixture is cooled to 4° C. and maintained for 3-4 hours. The solid is filtered and washed with acetone (20 mL). The wet solid (34.0 g), acetone (120 mL) and methanol (30 mL) are charged into a round-bottom flask and stirred for 10 minutes. The mixture is heated to 55° C. and stirred for 10 minutes. The solution is cooled to 4-5° C. and stirred for 3-4 hours. The formed solid is filtered, washed with a chilled mixture of acetone (8 mL) and methanol (2 mL), and dried at 75° C., to afford 26.0 g of the title compound.

Purity by HPLC: 99.809%; dimer impurity of Formula (Va): 0.042%.

### Example 24

### Preparation of Ranolazine

N-(2,6-dimethylphenyl)-1-piperazine acetamide (20 g), 1-(2-methoxyphenoxy)-2,3-epoxypropane (18.9 g) and acetone (100 mL) are charged at 26° C. into a round-bottom flask and stirred for 5 minutes. The mixture is heated to 54-55° C. and maintained for 11-12 hours. The mixture is cooled to 3-5° C. and maintained for 3-4 hours. The solid is filtered and washed with acetone (20 mL). The wet solid (30.9 g), acetone (120 mL) and methanol (30 mL) are charged into a round-bottom flask and stirred for 10 minutes. The mixture is heated to 56° C. and stirred for 30 minutes. The solution is cooled to 5° C. and stirred for 3-4 hours. The formed solid is filtered, washed with a chilled mixture of acetone (8 mL) and methanol (2 mL), and dried at 75° C., to afford 25.6 g of the title compound.

Purity by HPLC: 99.80%; compound of Formula (II): 0.002%; chloro impurity of Formula (IIb): 0.112%; dimer impurity of Formula (Va): 0.014%; compound of Formula (III): not detected; compound of Formula (IV): not detected; dimer impurity of Formula (IVa): 0.01%; dichloroacetyl impurity of Formula IIIa: not detected; ECH dimer impurity of Formula (VI): 0.008%.

### Example 25

### Preparation of Ranolazine

N-(2,6-dimethylphenyl)-1-piperazine acetamide (50.0 g), 1-(2-methoxyphenoxy)-2,3-epoxypropane (47.36 g) and methanol (50 mL) are charged into a round-bottom flask and stirred for 5 minutes. The mixture is heated to 65° C. and stirred for 1-2 hours. Acetone (200 mL) is added at 50° C. and stirred at 55° C. for 10 minutes. The mixture is cooled to 4° C. and stirred for 4-5 hours. The solid is filtered and washed with chilled acetone (50 mL). The wet solid, methanol (50 mL), and acetone (200 mL) are charged into a round-bottom flask and stirred for 5 minutes. The mixture is heated to 56° C. and stirred for 45 minutes, then cooled to 5° C. The mixture is stirred at 5° C. for 5-6 hours and the formed solid is filtered, washed with acetone (50 mL), and dried at 75° C., to afford 58.0 g of the title compound.

Purity by HPLC: 99.849%.

### Example 26

### Preparation of Ranolazine

N-(2,6-dimethylphenyl)-1-piperazine acetamide (20.0 g) and 142-methoxyphenoxy)-2,3-epoxypropane (18.9 g) are charged into a round-bottom flask and stirred for 5 minutes. The mixture is heated to 50° C. and maintained for 5 minutes. The mixture is heated to 68-72° C. and maintained for 30 minutes. Methanol (10 mL) is added and stirred for 5 minutes. Acetone (80 mL) is added and stirred at 58° C. for 10 minutes. The mixture is cooled to 3° C. and stirred for 3 hours. The solid is filtered and washed with acetone (20 mL). The solid is suction dried for 15 minutes. The wet solid, acetone (120 mL), and methanol (30 mL) are charged into a round-bottom flask and the mixture is heated to 53° C. The solution is maintained at 53° C. for 15 minutes then is cooled to 5° C. and stirred for 5-6 hours. The formed solid is filtered, washed with acetone (20 mL), and dried at 70° C., to afford 22.0 g of the title compound.

Purity by HPLC: 99.68%.

### Example 27

### Purification of Ranolazine

Ranolazine (20 g), acetone (80 mL) and methanol (20 mL) are charged into a round bottom flask and stirred for 5 minutes. The mixture is heated to 54° C. and maintained at reflux for 20 minutes. The mixture is cooled to 25-35° C., then further cooled to 2-5° C. and maintained for 3-4 hours. The precipitated solid is filtered under reduced pressure and washed with acetone (20 mL). The solid is dried at 70-73° C., to afford 16.5 g of the title compound.

Purity by HPLC: 99.93%; compound of Formula (II): 0.4 ppm; compound of Formula (III): less than 0.16 ppm; chloro impurity of Formula (IIb): 0.1 ppm.

Purity by GC: 2,6-dimethyl aniline of Formula (VII): not detected; epichlorohydrin of Formula (VIII): not detected.

1. A process for preparing ranolazine of Formula (I) or a pharmaceutically acceptable salt thereof, 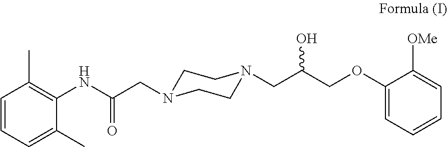
comprising reacting 2-methoxyphenol with epichlorohydrin, in the presence of a base, to provide 1-(2-methoxyphenoxy)-2,3-epoxypropane of Formula (II), 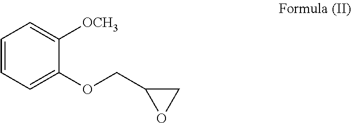
wherein the base is added to the reaction mixture in more than one portion.

2. The process of claim 1, wherein less than about 50 percent of the total amount of base is added in a single portion.

3. The process of claim 1, wherein a base comprises an alkali or alkaline metal hydroxide, or an ion exchange resin.

4. The process of claim 1, further comprising:
reacting 1-(2-methoxyphenoxy)-2,3-epoxypropane of Formula (II) with piperazine, provide 1-[3-(2-methoxyphenoxy)-2-hydroxypropyl]-piperazine of Formula (V); 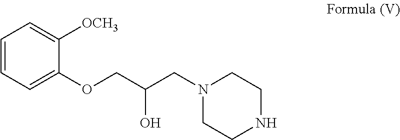
reacting 1-[3-(2-methoxyphenoxy)-2-hydroxypropyl]-piperazine of Formula (V) with [(2,6-dimethylphenyl)aminocarbonylmethyl]-chloride of Formula (III) to provide ranolazine of Formula (I); and 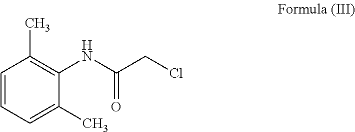
isolating ranolazine of Formula (I) in solid form.

5. The process of claim 1, further comprising:
reacting [(2,6-dimethylphenyl)aminocarbonylmethyl]-chloride of Formula (III) with piperazine, to form N-(2,6-dimethylphenyl)-1-piperazine acetamide of Formula (IV); 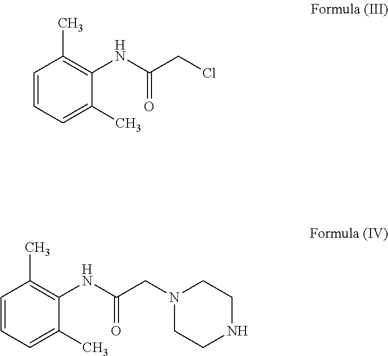
reacting 1-(2-methoxyphenoxy)-2,3-epoxypropane of Formula (II) with N-(2,6-dimethylphenyl)-1-piperazine acetamide of Formula (IV), to form ranolazine of Formula (I); and
isolating ranolazine of Formula (I) in solid form.

6. The process of claim 5, further comprising recrystallizing N-(2,6-dimethylphenyl)-1-piperazine acetamide of Formula (IV) to reduce a piperazine impurity content.

7. The process of claim 5, further comprising reducing a piperazine content in N-(2,6-dimethylphenyl)-1-piperazine acetamide of Formula (IV), prior to reacting with 1-(2-methoxyphenoxy)-2,3-epoxypropane of Formula (II), by a process comprising:
(a) providing a mixture containing N-(2,6-dimethylphenyl)-1-piperazine acetamide of Formula (IV) and a solvent;
(b) adjusting the pH to less than about 7 by adding an acid; and
(c) adjusting the pH to greater than about 8 by adding a base, and isolating N-(2,6-dimethylphenyl)-1-piperazine acetamide of Formula (IV), substantially free of piperazine.

8. The process of claim 7, wherein an acid comprises an organic acid.

9. The process of claim 7, wherein an acid comprises one or more of formic acid, acetic acid, benzoic acid, p-toluenesulphonic acid, methanesulphonic acid, phosphoric acid, and sulphuric acid.

10. The compound N-(2,6-dimethylphenyl)-1-piperazine acetamide of Formula (IV), substantially free of piperazine.

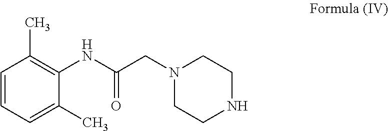

11. A process for purifying N-(2,6-dimethylphenyl)-1-piperazine acetamide of Formula (IV), comprising: 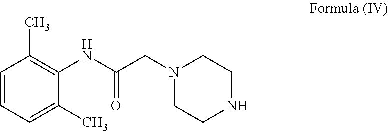
(a) providing a mixture containing N-(2,6-dimethylphenyl)-1-piperazine acetamide of Formula (IV) and a solvent;
(b) adjusting pH to less than about 7 with an acid;
(c) adjusting pH to greater than about 8 with a base; and
(d) isolating N-(2,6-dimethylphenyl)-1-piperazine acetamide of Formula (IV), substantially free of piperazine.

12. The process of claim 11, wherein an acid comprises an organic acid.

13. The process of claim 11, wherein an acid comprises one or more of formic acid, acetic acid, benzoic acid, p-toluenesulphonic acid, methanesulphonic acid, phosphoric acid, and sulphuric acid.

14. Ranolazine, substantially free of any one or more of impurities having the formulae:

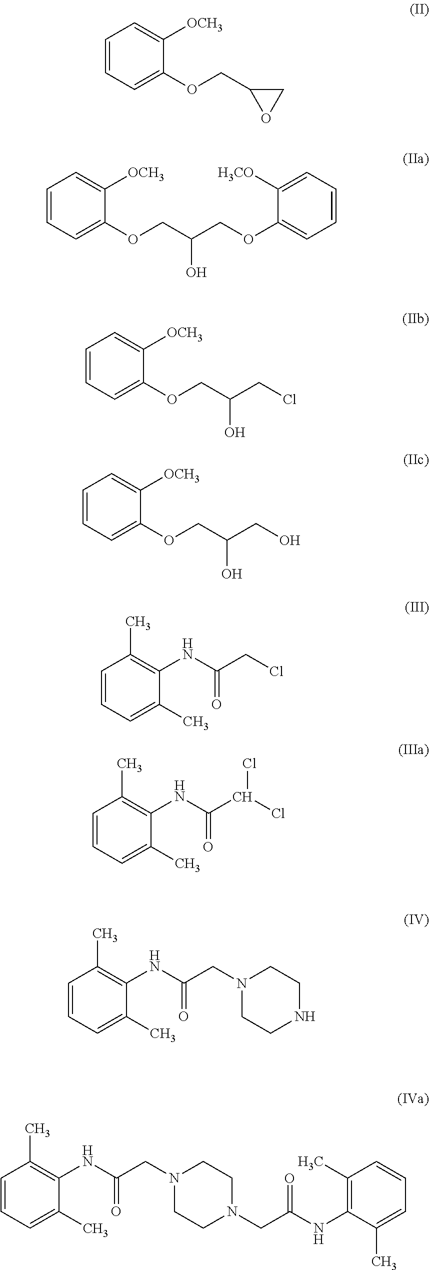

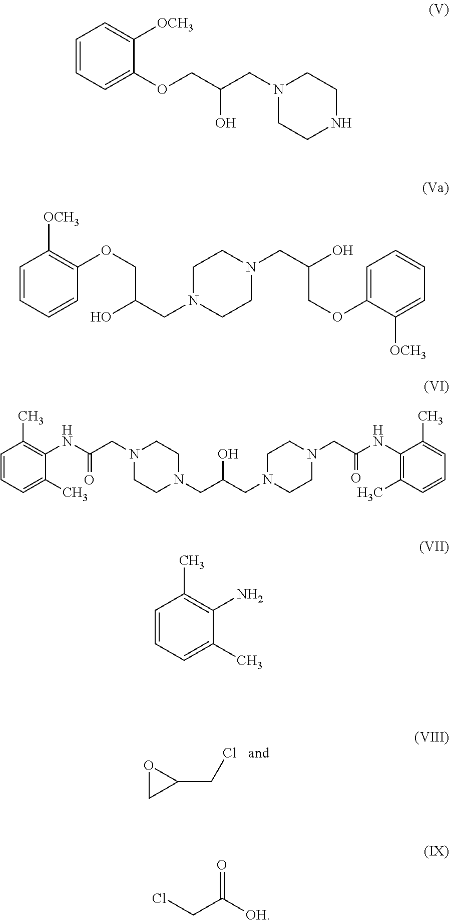

15. Ranolazine having particle sizes less than about 150 μm.

16. Ranolazine of claim 15 having particle sizes less than about 100 μm.

17. Ranolazine of claim 15 having particle sizes less than about 50 μm.

18. Ranolazine of claim 15 having particle sizes less than about 20 μm.

19. Ranolazine of claim 15 having particle sizes less than about 10 μm.

20. Ranolazine having a bulk density less than about 0.8 g/mL.

21. Ranolazine having a specific surface area greater than about 0.1 m²/g.
